(19) United States
(12) Patent Application Publication
(10) Pub. No.: US 2015/0025810 A1
(43) Pub. Date: Jan. 22, 2015

(54) ASSAY TO DETECT A GYNECOLOGICAL CONDITION

(71) Applicant: Healthlinx Limited, Richmond (AU)

(72) Inventors: Dominic J. Autelitano, Camberwell (AU); Tracey A. Edgell, Blind Bight (AU); Nick Gatsios, Essendon (AU); Leodevico L. Ilag, Balwyn (AU)

(73) Assignee: Healthlinx Limited, Richmond (AU)

(21) Appl. No.: 14/180,833

(22) Filed: Feb. 14, 2014

Related U.S. Application Data
(63) Continuation of application No. 12/988,622, filed on Oct. 19, 2010, now abandoned.

(30) Foreign Application Priority Data
Apr. 23, 2008 (AU) 2008902029
Oct. 1, 2008 (AU) 2008905120

(51) Int. Cl. G01N 33/574; G01N 33/68; G06F 19/18

(52) U.S. Cl. CPC G01N 33/57449; G06F 19/18; G01N 33/689
USPC 702/19

(57) ABSTRACT

The present invention relates generally to the field of diagnostic and prognostic assays for a gynecological condition. More particularly, the present invention provides an assay for diagnosing the presence of or a risk of having a gynecological cancer or a sub-type thereof or a stage of the cancer or complications arising therefrom or other gynecological condition including an inflammatory disorder.

### FILING DATA

This application is associated with and claims priority from Australian Patent Application No. 2008902029, filed on 23 Apr. 2008 and Australian Patent Application No. 2008905120, filed on 1 Oct. 2008, the entire contents of which are incorporated herein by reference.

### FIELD

The present invention relates generally to the field of diagnostic and prognostic assays for a gynecological condition. More particularly, the present invention provides an assay for diagnosing the presence of or a risk of having a gynecological cancer or a sub-type thereof or a stage of the cancer or complications arising therefrom or other gynecological condition including an inflammatory disorder. The assays of the present invention are capable of integration into pathology architecture to provide a diagnostic and reporting system.

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Ovarian cancer is one of the most lethal gynecologic malignancies and is the fifth most common cause of mortality in women. The single most important factor keeping the fatality levels high is the lack of early detection in the early treatable stages of disease.

During the early stages (stages I and II) of disease, the cancer is contained within the ovaries (stage I) or within the other organs of the pelvis (stage II). Detection of stage I disease has a greater than 80% survival rate at 5 years, dropping to over 70% for stage II. At its later stages, the cancer has spread beyond the pelvis to the lining of the abdomen or lymph nodes. At this point, the 5 year survival rate post detection is reduced to less than 50%. The final most advanced stage of this disease is stage IV by which point metastasis to the liver, lungs or other organs has occurred, and survival is less than 30%.

Generally early-stage ovarian cancer is asymptomatic, and the majority of the diagnoses are made at a time when the disease has already established regional or distant metastases. Despite aggressive cytoreductive surgery and platinum-based chemotherapy, the 5-year survival for patients with clinically advanced ovarian cancer is only 15 to 20 percent, although the cure rate for stage I disease is usually greater than 90 percent (Holschneider and Berek, Semin Surg Oncol. 19 (1):3-10, 2000). These statistics provide the primary rationale to improve ovarian cancer screening and early identification.

The mortality rates associated with ovarian cancer are high in part because of a lack of effective early detection methods. If detected early, survival is dramatically increased. Research has focused on developing improved ways of evaluating women, particularly those at high risk, for the first signs of ovarian cancer. As yet, however, a premalignant lesion has not been identified. Although alterations of several genes, such as c-erb-B2, c-myc, and p53, have been identified in a significant fraction of ovarian cancers, none of these mutations is diagnostic of malignancy or predictive of tumor behavior over time (Veikkola et al, Cancer Res 60 (2):203-12, 2000; Berek et al, Am J Obstet Gynecol, 164 (4):1038-42, 1991; Cooper et al, Clin Cancer Res. 8 (10):3193-7, 2002; and Di Blasio er al, J Steroid Biochem Mol Biol. 53 (1-6):375-9, 1995). Instead, high-risk women must rely on genetic counseling and testing, as well as measurement of serum CA125 level and transvaginal ultrasound (Oehler and Caffier, Anticancer Res, 20 (6D):5109-12, 2000; Santin et al, Eur J Gynaecol Onco 20 (3):177-81, 1999; and Senger et al, Science 219 (4587):983-5, 1983). However, CA125 is neither sensitive nor specific for detecting early stage disease. CA125, therefore, is not suitable for general screening. It is only thought to be robust in monitoring the response or progression of the disease, but not as a diagnostic or prognostic marker (Gadducci et al, Anticancer Res 19 (2B):1401-5, 1999).

Screening using transvaginal ultrasound, Doppler and morphological indices has shown some encouraging results but, used alone, it currently lacks the specificity required of a screening test for the general population (Karayiannakis et al, Surgery 131 (5):548-55, 2002 and Lee et al, Int J Oncol 17 (1): 149-52, 2000). Combinational multimodal screening using tumor markers and ultrasound yields higher sensitivity and specificity. This combination approach is also the most cost-effective potential screening strategy (Karayiannakis et al, 2002 supra and Lee et al, 2000 supra). However, it too is of questionable effectiveness in the general population. Thus, there is a critical need to develop additional markers for early detection of disease.

It has been proposed that improved specificity and sensitivity may be achieved by using serum/plasma protein markers in combination with CA125.

Gorelik et al, Cancer Epidemiol, Biomarkers Prev 14(4):981-987, 2005, used a multiplex assay design with a final classification tree analysis to discriminate control groups from ovarian cancer. Their multiplex design used CA125 in combination with inter alia EGF and VEGF, and reported an improved sensitivity level of 90-100% at a specificity of 80-90%, as compared to the CA125 marker alone which achieved only 70-80%.

In similar vein, Visintin at al, Clin Cancer Res 14(4):1065-1072, 2008, have reported a study in which both multiplex and ELISA were used to test healthy controls and ovarian cancer patients based on a panel of markers. Their elected markers were CA125 combined with leptin, prolactin, osteopontin, insulin-like growth factor II, and macrophage inhibitory factor. Whilst none of the biomarkers by itself was able to discriminate between disease and control, the combination achieved 84-98% sensitivity at a specificity of 95%, as compared to the CA125 alone which achieved only 72% sensitivity at the same level of specificity.

There is a need to develop a highly sensitive assay for gynecological conditions such as ovarian cancer and complications therefrom and in particular early stage ovarian cancer as well as other gynecological conditions including inflammatory disorders.

### SUMMARY

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

A method for the detection and monitoring of a gynecological condition such as a gynecological cancer is provided. The term “gynecological condition” includes complications arising from a gynecological cancer as well as an inflammatory disorder such as endometriosis. The method herein particularly enables early stage detection of a gynecological condition, facilitates histological examination and permits monitoring of therapeutic regimens. The present invention is particularly useful when applied to the diagnosis of symptomatic women, but may equally be applied to the diagnosis of asymptomatic women and/or women at high risk of developing a gynecological condition. One aspect of the method of the present invention is a proteomic and in a particular embodiment, a multifactorial assay in which the levels of combinations of two or more biomarkers or analytes selected from the list comprising anterior gradient protein-2 (AGR-2), midkine, CA125, interleukin-6 (1H-6), interleukin-8 (IL-8), C-reactive protein (CRP), serum amyloid A (SAA) and serum amyloid P (SAP) are detected. Reference to these biomarkers and in particular AGR-2, midkine, CA125, TL-6, IL-8, CRP, SAA and SAP includes any derivatives or modified forms thereof such as polymorphic variants, truncated forms, aggregated or multimeric forms as well as homologs thereof. The assay of the present invention is particularly adaptable for integration into pathology platforms or architecture.

In one embodiment, the relative alteration in the concentrations of the two or more biomarkers compared to a control is indicative of a gynecological disease condition or the level of response to therapy. In another embodiment, the levels are subjected to multivariate analysis to create an algorithm which enables the determination of an index of probability of the presence or absence of the condition. In another aspect, the detection of an altered level in concentration of AGR-2 or midkine alone or in combination with other markers including CA125 is indicative of a gynecological condition. Reference to “altered” includes an increase or decrease in concentration of the biomarkers in tissues or fluid such as plasma relative to a control sample or threshold level or a database of standard normal values or following algorithmic analysis. Generally, the alteration is an increase in concentration of the biomarkers.

Notwithstanding the proteomic approach, the present invention extends to a genetic approach to measure expression of genes encoding the above-mentioned biomarkers.

The biomarker concentrations (i.e. levels) of two or more of the biomarkers provides a measurable relationship between biomarker levels and disease status in patients. In addition to “level” of biomarker, the present invention extends to ratios of two or more markers as input data for comparison to controls or for multivariate analysis leading to an algorithm. The present invention extends to the detection of a gynecological condition by screening for an altered level in the concentration of AGR-2 or midkine alone or in combination with CA125. Hence, an altered level in AGR-2 or midkine concentration alone or in combination with CA 125 or other biomarkers is indicative of a condition. Alternatively, the level of AGR-2 or midkine alone or in combination with other biomarkers may be used in the multifactorial, algorithm approach.

The selected biomarkers may also be used collectively or individually in histological assessment of tissue or to monitor the efficacy of a treatment regime. The biomarkers are also useful to sub-type a gynecological cancer or to determine the stage of the cancer which may influence the type of anti-cancer therapy employed. Hence, the present invention extends to a personalized medicine approach to treat a gynecological cancer. The present invention extends to other gynecological conditions such as inflammatory disorders.

Accordingly, one aspect of the present invention contemplates an assay for determining the presence of a gynecological condition in a subject, the assay comprising determining the concentration of two or more of AGR-2, midkine and/or CA125 or modified or homolog forms thereof in a biological sample from the subject wherein an altered level in two or more of AGR-2, midkine and/or CA125 or their modified or homolog forms is indicative of the subject having a gynecological condition. Levels of AGR-2 or midkine or CA125 or their modified or homolog forms may also be screened alone or in combination with other biomarkers. As indicated above, the term “altered” means an increase or elevation in concentration or a decrease or reduction in concentration. Testing may be in tissue, tissue fluid or blood including plasma or serum.

More particularly the present invention provides, an assay for determining the presence of a gynecological condition in a subject, the assay comprising determining levels of biomarkers in a biological sample from the subject wherein the biomarker is CA125 and at least one selected from AGR-2, midkine and CRP or modified or homolog forms thereof wherein an alteration in the levels of the biomarkers relative to a control is indicative of the presence of the subject having or not having the condition.

In an alternative embodiment, the present invention provides an assay for determining the presence of a gynecological condition in a subject, the assay comprising determining the concentration of biomarkers in a biological sample from the subject, the biomarkers selected from two or more of AGR-2, midkine and CA125 or modified or homolog forms thereof; two or more of CA125, IL-6, IL-8, CRP, SAA and SAP or modified or homolog forms thereof; two or more of IL-6, IL-8, CRP, SAA and SAP or modified or homolog forms thereof; or at least one of CA125, IL-6, IL-8, CRP, SAA and SAP or modified or homolog forms thereof, and at least one of midkine or AGR-2 or modified or homolog forms thereof; subjecting the concentrations to an algorithm generated from a first knowledge base of data comprising the levels of the same biomarkers from a subject of known status with respect to the condition wherein the algorithm provides an index of probability of the subject having or not having the condition.

Hence, in one embodiment, the present invention provides a diagnostic rule based on the application of a comparison of levels of biomarkers to control samples. In another embodiment, the diagnostic rule is based on application of statistical and machine learning algorithms. Such an algorithm uses the relationships between biomarkers and disease status observed in training data (with known disease status) to infer relationships which are then used to predict the status of patients with unknown status. Practitioners skilled in the art of data analysis recognize that many different forms of inferring relationships in the training data may be used without materially changing the present invention.

In an embodiment, the condition is a cancer such as ovarian cancer or a complication arising therefrom. In another embodiment, the condition is a gynecological inflammatory condition such as but not limited to endometriosis.

Determining the “presence” of a condition includes determining a risk of having a condition. A “risk” is conveniently considered in terms of determining an index of probability of having a condition relative to a subject who does not have the condition.

Hence, the present invention contemplates the use of a knowledge base of training data comprising levels of biomarkers from a subject with a gynecological condition, upon input of a second knowledge base of data comprising concentrations of the same biomarkers from a patient with an unknown gynecological condition, provides an index of probability that predicts the nature of the gynecological condition or the absence of the condition.

The present invention further contemplates an assay for detecting ovarian cancer in a subject, the assay comprising contacting a sample from the subject with an immobilized ligand to two or more of AGR-2, midkine or CA125 or modified or homolog forms thereof for a time and under conditions for AGR-2 or midkine or CA125 or modified or homolog forms thereof to bind to its ligand which provides an indication of the concentration of AGR-2, midkine and/or CA125 or modified or homolog forms thereof wherein an altered concentration of two or more of AGR-2, midkine and/or CA125 or modified or homolog forms thereof is indicative of ovarian cancer.

In an alternative embodiment, the present invention contemplates an assay for detecting ovarian cancer in a subject, the assay comprising contacting a sample from the subject with immobilized ligands to two or more of AGR-2, midkine and/or CA125 or modified or homolog forms thereof; two or more of CA125, IL-6, IL-8, CRP, SAA and/or SAP or modified or homolog forms thereof; two or more of IL-6, IL-8, CRP, SAA and/or SAP or modified or homolog forms thereof; or at least one of CA125, IL-6, IL-8, SAA and/or SAP or modified or homolog forms thereof and at least one of midkine and/or AGR-2 alone or in combination with CA125 or modified or homolog forms thereof for a time and under conditions sufficient for the biomarker to bind to a ligand and then detecting the level of binding which is indicative of the concentration of the biomarker and subjecting the concentrations to an algorithm generated using levels of biomarkers in a subject having ovarian cancer to provide an index of probability that the subject has or does not have ovarian cancer.

Another aspect of the present invention is directed to a panel of ligands to biomarkers useful in the detection of a gynecological condition, the panel comprising ligands to two or more of AGR-2, midkine and/or CA125 or modified or homolog forms thereof; two or more of CA125, IL-6, IL-8, CRP, SAA or SAP or modified or homolog forms thereof; two or more of IL-6, IL-8, CRP, SAA or SAP; or modified or homolog forms thereof or at least one of CA125, IL-6, IL-8, CRP, SAA or SAP or modified or homolog forms thereof and at least one of midkine or AGR-2 alone or in combination with CA 125 or modified or homolog forms thereof.

In particular, the present invention provides a panel of biomarkers for the detection of a gynecological condition in a subject, the panel comprising agents which bind specifically to biomarkers, the biomarkers selected from two or more of AGR-2, midkine and/or CA125 or modified or homolog forms thereof; two or more of CA125, IL-6, IL-8, CRP, SAA and SAP or modified or homolog forms thereof; two or more of IL-6, IL-8, CRP, SAA and SAP or modified or homolog forms thereof, and at least one of CA125, IL-6, IL-8, CRP, SAA and SAP or modified or homolog forms thereof and at least one of midkine or AGR-2 alone or in combination with CA125 or modified or homolog forms thereof to determine the levels of two or more biomarkers and then subjecting the levels to an analysis to determine any alteration such as an increase in biomarker levels.

In an embodiment, the concentrations are subjected to comparison to a control or database of “normal” or “abnormal” values. In another embodiment, the concentrations are subjected to an algorithm generated from a first knowledge base of data comprising the levels of the same biomarkers from a subject of known status with respect to the condition wherein the algorithm provides an index of probability of the subject having or not having the condition.

Still another aspect of the present invention contemplates a kit for diagnosing the presence or absence of a gynecological condition, the kit comprising a composition of matter comprising the elements [X]ₙ, Y and [Z]ₘ wherein:

X is a ligand to a biomarker selected from CA125 or modified or homolog forms thereof and n is 0 or 1;
Y is a ligand to a biomarker selected from the list comprising, when n is 0, one or more of AGR-2 and/or midkine or modified or homolog forms thereof; two or more of IL-6, IL-8, CRP, SAA and SAP or modified or homolog forms thereof or when ₙ is 1, at least one of IL-6, IL-8, CRP, SAA and SAP or modified or homolog forms thereof; and
Z is a ligand to a biomarker selected from midkine and AGR-2 or modified or homolog forms thereof and m is 0 or 1;
the kit further comprising reagents to facilitate determination of the concentration of biomarker binding to a ligand. In use, the kit facilitates the determination of biomarker levels. These levels can be compared to a control or database of values. In another embodiment, the levels are subjected to an algorithm generated from a first knowledge base of data comprising the levels of the same biomarkers from a subject of known status with respect to the condition wherein the algorithm provides an index of probability of the subject having or not having the condition.

The present invention further provides a panel of markers comprising the list [X]ₙ, [Y]ₓ and [Z]ₘ wherein:

X is CA125 or modified or homolog forms thereof and n is 0 or 1;
Y is a marker selected from IL-6, IL-8, CRP, SAA and SAP or modified or homolog forms thereof provided that when n is 0, Y comprises two or more of the markers wherein x is 0 or 1; and
Z is two or more of AGR-2 or midkine and/or CA125 or modified or homolog forms thereof and m is 0 or 1.

Kits and knowledge-based computer software and hardware also form part of the present invention.

In particular, the assays of the present invention may be used in existing knowledge-based architecture or platforms associated with pathology services. For example, results from the assays are transmitted via a communications network (e.g. the internet) to a processing system in which an algorithm is stored and used to generate a predicted posterior probability value which translates to the index of disease probability which is then forwarded to an end user in the form of a diagnostic or predictive report.

The assay may, therefore, be in the form of a kit or computer-based system which comprises the reagents necessary to detect the concentration of the biomarkers and the computer hardware and/or software to facilitate determination and transmission of reports to a clinician.

The assay of the present invention permits integration into existing or newly developed pathology architecture or platform systems. For example, the present invention contemplates a method of allowing a user to determine the status of a subject with respect to a gynecological cancer or subtype thereof or stage of cancer, the method including:
(a) receiving data in the form of levels or concentrations of CA125 and one or more of AGR-2, midkine, CRP, IL-6, IL-8, SAA and SAP from the user via a communications network;
(b) processing the subject data via multivariate analysis to provide a disease index value;
(c) determining the status of the subject in accordance with the results of the disease index value in comparison with predetermined values; and
(d) transferring an indication of the status of the subject to the user via the communications network reference to the multivariate analysis includes an algorithm which performs the multivariate analysis function.

Conveniently, the method generally further includes:
(a) having the user determine the data using a remote end station; and
(b) transferring the data from the end station to the base station via the communications network.

The base station can include first and second processing systems, in which case the method can include:
(a) transferring the data to the first processing system;
(b) transferring the data to the second processing system; and
(c) causing the first processing system to perform the multivariate analysis function to generate the disease index value.

The method may also include:
(a) transferring the results of the multivariate analysis function to the first processing system; and
(b) causing the first processing system to determine the status of the subject.

In this case, the method also includes at lest one of:
(a) transferring the data between the communications network and the first processing system through a first firewall; and
(b) transferring the data between the first and the second processing systems through a second firewall.

The second processing system may be coupled to a database adapted to store predetermined data and/or the multivariate analysis function, the method include:
(a) querying the database to obtain at least selected predetermined data or access to the multivariate analysis function from the database; and
(b) comparing the selected predetermined data to the subject data or generating a predicted probability index.

The second processing system can be coupled to a database, the method including storing the data in the database.

The method can also include having the user determine the data using a secure array, the secure array of elements capable of determining the level of biomarker and having a number of features each located at respective position(s) on the respective code. In this case, the method typically includes causing the base station to:
(a) determine the code from the data;
(b) determine a layout indicating the position of each feature on the array; and
(c) determine the parameter values in accordance with the determined layout, and the data.

The method can also include causing the base station to:
(a) determine payment information, the payment information representing the provision of payment by the user; and
(b) perform the comparison in response to the determination of the payment information.

The present invention also provides a base station for determining the status of a subject with respect to a gynecological cancer or a subtype thereof or a stage of the cancer, the base station including:
(a) a store method;
(b) a processing system, the processing system being adapted to:
   (i) receive subject data from the user via a communications network, the data including levels or concentrations of two or more biomarkers selected from AGR-2, midkine, CA125, IL-6, IL-8, CRP, SAA and SAP from a subject;
   (ii) performing an algorithmic function including comparing the data to predetermined data;
   (iii) determining the status of the subject in accordance with the results of the algorithmic function including the comparison; and
(c) output an indication of the status of the subject to the user via the communications network.

The processing system can be adapted to receive data from a remote end station adapted to determine the data.

The processing system may include:
(a) a first processing system adapted to:
   (i) receive the data; and
   (ii) determine the status of the subject in accordance with the results of the multivariate analysis function including comparing the data; and
(b) a second processing system adapted to:
   (i) receive the data from the processing system;
   (ii) perform the multivariate analysis function including the comparison; and
   (iii) transfer the results to the first processing system.

The base station typically includes:
(a) a first firewall for coupling the first processing system to the communications network; and
(b) a second firewall for coupling the first and the second processing systems.

The processing system can be coupled to a database, the processing system being adapted to store the data in the database.

Yet another aspect of the present invention is directed to the use of the levels of two or more biomarkers selected from AGR-2, midkine, CA125, IL-6, IL-8, CRP, SAA and SAP or modified or homolog forms thereof, to detect ovarian cancer or other gynecological condition in a subject.

Still another aspect of the present invention provides the use of levels of AGR-2 or midkine or modified or homolog forms thereof in the generation of an assay to detect ovarian cancer or other gynecological condition in a subject.

Even another aspect of the present invention provides the use of levels of AGR-2, midkine and CA125 or modified or homolog forms thereof in the generation of an assay to detect ovarian cancer or other gynecological condition in a subject.

### BRIEF DESCRIPTION OF THE FIGURES

FIG. 1 is a diagrammatical representation of the modeling to provide an algorithm which generates an index of probability that a subject has or does not have a gynecological condition.

FIG. 2 is a diagrammatical representation showing both modeling and validation of biomarker data.

FIGS. 3 a and b are schematic representations of the assay of the present invention linked to a pathology platform to provide a report on the index of disease probability of a subject having or not having a gynecological cancer.

FIGS. 4 and 5 are schematic representations of the assay linked to a pathology platform to provide a report. 1, end station; 2 base station; 3, client serve (e.g. a simple object application protocol (SOAP); 4, communications network (e.g. internet); LIMS, Laboratory Information Management system; an example of an assay report is shown in FIG. 6.

FIG. 6 is a data representation of a report generated by the assay shown in FIG. 3.

FIG. 7 is a photographical representation showing immunohistochemical localization of immunoreactive (ir)-AGR-2 in sections of normal human ovary. Normal ovarian epithelium (arrows) was consistently negative for ir-AGR-2 (A,B). Small inclusion cysts within normal ovary demonstrated occasional cells (arrows) with distinct cytoplasmic staining for ir-AGR-2 (D). Magnification is ×200 for A, C and ×400 for B,D.

FIG. 8 is a photographical representation showing immunohistochemical localization of ir-AGR-2 in epithelial cell-derived ovarian tumors. (A) Benign mucinous tumor of endocervical type. Virtually all of the epithelium displays strong granular cytoplasm staining. Staining is particularly intense basally and along the cell membranes. (B) A serous borderline tumor with epithelial cells exhibiting strong granular staining of varying intensity. (C) Well differentiated Grade 1 endometrioid tumor with a well developed glandular pattern. The tumor exhibits strong granular cytoplasm staining of groups of cells throughout the epithelium. In many cells, staining appears more intense along the cell/cell membranes and apical surface. (D) Grade 1 endometrioid tumor with a well differentiated glandular pattern. The tumor exhibits dense granular cytoplasmic staining of variable intensity within the glands. (E) Grade 2 serous tumor. An island of well-defined immunoreactive cells are present within a largely negatively staining, moderately differentiated tumor. The staining is granular, occupies most of the cytoplasm and is more densely accumulated near the apex. (F) A predominantly poorly differentiated Grade 3 serous tumor with scattered groups of isolated cells exhibiting strong, dense, granular staining for ir-AGR-2. (G) Grade 3 serous tumor section showing a remnant, well differentiated, strongly immunostaining gland adjacent to a poorly differentiated grade 3 tumor. (H) A serous Grade 3 carcinoma with a papillary pattern exhibiting strong cytoplasm immunostaining of groups of tumor cells lining the papillae. (I); Grade 3 clear cell carcinoma showing a typical clear cell pattern. There is extensive cytoplasmic immunostaining of cells within the tumor nests and cords. (Magnification ×200 for C, E, G and I and ×400 for A, B, D, F and H).

FIG. 9 is a photographic representation of a Western blot of pooled human plasma samples using affinity purified rabbit anti-AGR-2 (1:500). Individual plasma samples (3-6 per group) were obtained from control subjects and from patients with diagnosed serous, mucinous and clear cell ovarian carcinoma of various grades. Equivalent amounts of individual plasma samples in each group were pooled and depleted of the top six plasma proteins using Multiple Affinity Removal System (Agilent) to concentrate remaining plasma proteins and enhance detection. The equivalent of 12 μg of depleted plasma protein from each group was then Western blotted using anti-AGR-2 using chemiluminesence detection. A weak immunoreactive species of approximately 18 kDa (mature AGR-2) is evident in mucinous and clear cell ovarian carcinoma plasma, but not in control plasma or plasma derived from serous ovarian cancer patients, suggesting differential expression and secretion of ir-AGR-2 associated with different ovarian tumor types. A number of higher molecular weight immunoreactive species are also labeled with the anti-AGR-2 antibody. These species similarly appear to be differentially expressed in plasma samples derived from patients with different ovarian tumor types.

FIG. 10 is a graphical representation of the ROC curve analysis described in Table 10, obtained with the model sample subset, comparing CA125 and the biomarker panel shown in Table 9.

FIG. 11 is a graphical representation of the ROC curve analysis described in Table 12, obtained with the validation sample subset, comparing CA125 and the biomarker panel shown in Table 11.

FIG. 12 is a graphical representation of the ROC curve analysis described in Table 14, obtained with the entire sample set comparing CA125 and the biomarker panel shown in Table 13.

FIG. 13 is a graphical representation of the ROC curve analysis described in Table 17, obtained with the model sample subset comparing CA125 and the biomarker panel shown in Table 9.

FIG. 14 is a graphical representation of the ROC curve analysis described in Table 18, obtained with the validation sample subset comparing CA 25 and the biomarker panel shown in Table 11.

FIG. 15 is a graphical representation of the ROC curve analysis described in Table 19, obtained with the entire sample set comparing CA125 and the biomarker panel shown in Table 13.

FIG. 16 is a graphical representation of the mean concentration+/−SEM of AGR-2 in early stage ovarian cancer patients versus normal samples.

FIG. 17 is a graphical representation of mean plasma concentration±SEM of AGR-2 in early stage (Stage I/II) ovarian cancer patients versus Control samples.

FIG. 18 is a graphical representation of the correlation between plasma concentrations of AGR-2 and CA 125 in early stage (Stage I/II) ovarian cancer patients and healthy controls.

FIG. 19 is a graphical representation of the ROC curve analysis described in Table 21 for both CA125 and AGR-2 individually and as a two marker panel.

FIG. 20 is a graphical representation of plasma concentrations of AGR-2 in ovarian cancer patients versus controls. The bars represent the mean±SEM of 61 control and 46 ovarian cancer plasma samples (all cases), 35 of the ovarian cancer samples represented early stage (Stage I/II) disease. *P<0.05 vs Control.

FIG. 21 is a graphical representation of the mean±SEM plasma concentrations of AGR-2 in ovarian cancer patients versus controls (0, control; 1, serous type OVCA; 2, endometrioid; 3, mucinous; 4, mullerian mixed type; 5, clear cell).

### DETAILED DESCRIPTION

As used in the subject specification, the singular forms “a”, “an” and “the” include plural aspects unless the context clearly dictates otherwise. Thus, for example, reference to “a biomarker” includes a single biomarker, as well as two or more biomarkers; reference to “an analyte” includes a single analyte or two or more analytes; reference to “the invention” includes single and multiple aspects of the invention; and so forth.

The use of numerical values in the various ranges specified in this application, unless expressly indicated otherwise, are stated as approximations as though the minimum and maximum values within the states ranges were both preceded by the word “about”. In this manner, slight variations above and below the stated ranges can be used to achieve substantially the same results as values within the ranges. Also, the disclosure of these ranges is intended as a continuous range including every value between the minimum and maximum values. In addition, the present invention extends to ratios of two or more markers providing a numerical value associated with a level of risk of ovarian cancer development or presence.

A rapid, efficient and sensitive assay is provided for the identification of a gynecological condition. The gynecological condition includes cancer such as ovarian cancer or complications arising from cancer or inflammatory conditions such as endometriosis. In a particular embodiment, the assay enables early detection of ovarian cancer. Notwithstanding, the present invention is not limited to just the early detection of ovarian cancer since the assay may be used at any stage of a gynecological disease or its treatment or any complication arising therefrom.

Reference to a “cancer” with respect to a “gynecological condition” includes ovarian cancer as well as a sub-type of ovarian cancer such as mucinous or endometrial ovarian cancer or a stage of ovarian cancer such as stage I, II, III or IV. Terms such as “ovarian cancer”, “epithelial ovarian cancer” and an “ovarian malignancy” may be used interchangeably herein. The present invention is particularly useful when applied to the diagnosis of symptomatic women, but may equally be applied to the diagnosis of asymptomatic women and/or women at high risk of developing a gynecological condition.

Identified below are cytokine or analyte biomarkers useful in the detection of the gynecological condition and in particular ovarian cancer or a complication arising therefrom or a gynecological inflammatory condition. Collectively, these are referred to as “biomarkers” or “gynecological condition markers” or “markers of a gynecological condition”.

In one embodiment, the biomarkers are selected from two or more of AGR-2, midkine and/or CA125. In another embodiment two or more of IL-6, IL-8, CRP, SAA and/or SAP. In another embodiment, the biomarkers are selected from CA 125 and one or more of IL-6, IL-8, CRP, SAA and/or SAP. In yet another embodiment, the biomarkers include optionally CA125, two or more of IL-6, IL-8, CRP, SAA and/or SAP and wherein at least one of the latter biomarkers may be substituted by one or more of midkine or AGR-2. Notwithstanding, the present invention extends to replacing any one or more of the biomarkers with another analyte which, collectively or individually, assist in the detection of a gynecological condition. In addition, reference to any one or more of AGR-2, midkine, CA125, IL-6, IL-8, CRP, SAA and SAP includes a modified or homolog form thereof. A modified form includes a derivative, polymorphic variant, truncated form (truncate) and aggregated or multimeric forms or forms having expansion elements (e.g. amino acid expansion elements). For brevity, such modified and homolog forms are included by reference to any or some or all of the biomarkers.

Hence, the biomarkers represent a panel of markers comprising the list [X]ₙ, [Y]ₓ and [Z]ₘ wherein:

### X is CA125 and n is 0 or 1;

Y is a marker selected from IL-6, IL-8, CRP, SAA and SAP provided that when n is 0, Y comprises two or more of the markers wherein x is 0 or 1; and
Z is two or more of AGR-2, midkine and/or CA125 and m is 0 or 1.

Accordingly, one aspect of the present invention provides an assay for determining the presence of a gynecological condition in a subject, the assay comprising determining the concentration of biomarkers in a biological sample from the subject selected from two or more of AGR-2, midkine, CA125; two or more of CA125, IL-6, IL-8, CRP, SAA and SAP; two or more of IL-6, IL-8, CRP, SAA and SAP; or at least one of CA125, IL-6, IL-8, CRP, SAA and SAP and at least one of midkine or AGR-2; wherein an alteration in the levels of the biomarkers relative to a control provides an indication of the presence of the gynecological condition.

In an alternative embodiment, the present invention contemplates an assay for determining the presence of a gynecological condition in a subject, the assay comprising determining the concentration of biomarkers in a biological sample from the subject selected from two or more of AGR-2, midkine and/or CA125; two or more of CA125, IL-6, IL-8, CRP, SAA and/or SAP; two or more of IL-6, IL-8, CRP, SAA and/or SAP; or at least one of CA125, IL-6, IL-8, CRP, SAA and/or SAP and at least one of midkine and/or AGR-2; subjecting the levels to an algorithm generated from a first knowledge base of data comprising the levels of the same biomarkers from a subject of known status with respect to the condition wherein the algorithm provides an index of probability of the subject having or not having the condition. Reference to the “algorithm” is an algorithm which performs a multivariate analysis function.

In an alternative embodiment, the present invention contemplates an assay for determining the presence of a gynecological condition in a subject, the assay comprising determining the concentration of AGR-2 in a biological sample from the subject wherein an altered concentration in AGR-2 is indicative of the subject having a gynecological condition. In accordance with this embodiment, levels of AGR-2 may be screened alone or in combination with other biomarkers.

In an alternative embodiment, the present invention contemplates an assay for determining the presence of a gynecological condition in a subject, the assay comprising determining the concentration of midkine in a biological sample from the subject wherein an altered concentration in midkine is indicative of the subject having a gynecological condition. In accordance with this embodiment, levels of midkine may be screened alone or in combination with other biomarkers.

The latter three aspects of the invention may further involve determining the concentration of CA125.

In a particular embodiment, the gynecological condition is ovarian cancer or a complication arising therefrom or a stage of ovarian cancer such as Stage I or II or III or IV.

In another embodiment, the present invention provides an assay for determining the presence of ovarian cancer in a subject, the assay comprising determining levels of biomarkers in a biological sample from the subject selected from two or more of AGR-2, midkine and CA125; two or more of CA125, IL-6, IL-8, CRP, SAA and SAP; two or more of IL-6, IL-8, CRP, SAA and SAP; or at least one of CA125, IL-6, IL-8, CRP, SAA and SAP and at least one of midkine or AGR-2; wherein an alternative in the concentration of the biomarkers is indicative of the presence of the ovarian cancer.

Another aspect of the present invention contemplates an assay for determining the presence of ovarian cancer in a subject, the assay comprising determining levels of biomarkers in a biological sample from the subject selected from two or more of AGR-2, midkine, CA125; two or more of CA125, IL-6, IL-8, CRP, SAA and SAP; two or more of IL-6, IL-8, CRP, SAA and SAP; or at least one of CA125, IL-6, IL-8, CRP, SAA and SAP and at least one of midkine or AGR-2; subjecting the levels to an algorithm generated from a first knowledge base of data comprising the levels of the same biomarkers from a subject of known status with respect to the condition wherein the algorithm provides an index of probability of the subject having or not having the condition.

The first knowledge base of data may also come from multiple subjects.

In another embodiment, the present invention contemplates an assay for determining the presence of an ovarian cancer in a subject, the assay comprising determining the concentration of AGR-2 or midkine in a biological sample from the subject wherein an altered concentration in AGR-2 or midkine is indicative of the subject having an ovarian cancer. In accordance with this embodiment, levels of AGR-2, midkine or may be screened alone or in combination with other biomarkers. An “altered” level means an increase or elevation or a decrease or reduction in the concentrations of AGR-2 or midkine.

This aspect may also comprise determining the concentration of CA 125.

The determination of the concentrations or levels of the biomarkers enables establishment of a diagnostic rule based on the concentrations relative to controls. Alternatively, the diagnostic rule is based on the application of a statistical and machine learning algorithm. Such an algorithm uses relationships between biomarkers and disease status observed in training data (with known disease status) to infer relationships which are then used to predict the status of patients with unknown status. An algorithm is employed which provides an index of probability that a patient has a gynecological condition. The algorithm performs a multivariate analysis function.

Hence in one embodiment, the present invention provides a diagnostic rule based on the application of statistical and machine learning algorithms. Such an algorithm uses the relationships between biomarkers and disease status observed in training data (with known disease status) to infer relationships which are then used to predict the status of patients with unknown status. Practitioners skilled in the art of data analysis recognize that many different forms of inferring relationships in the training data may be used without materially changing the present invention.

Hence, the present invention contemplates the use of a knowledge base of training data comprising levels of biomarkers from a subject with a gynecological condition to generate an algorithm which, upon input of a second knowledge base of data comprising levels of the same biomarkers from a patient with an unknown gynecological condition, provides an index of probability that predicts the nature of the gynecological condition.

Alternatively, altered levels of AGR-2 is indicative of a gynecological condition.

Alternatively, altered levels of midkine is indicative of a gynecological condition.

The latter two aspects may also be in combination with altered levels of CA125.

The “subject” is generally a human female. However, the present invention extends to veterinary applications. Hence, the subject may be a non-human female mammal such as a bovine, equine, ovine animal or a non-human primate. Notwithstanding, the present invention is particularly applicable to detecting a gynecological cancer in a human female.

The term “training data” includes knowledge of levels of biomarkers relative to a control. A “control” includes a comparison to levels of biomarkers in a subject devoid of the gynecological condition or cured of the condition or may be a statistically determined level based on trials. The term “levels” also encompasses ratios of levels of biomarkers.

The “training data” also include the concentration of one or more of AGR-2, and/or midkine. The data may comprise information on an increase or decrease in AGR-2, and/or midkine concentration.

The present invention further contemplates a panel of biomarkers for the detection of a gynecological condition in a subject, the panel comprising agents which bind specifically to biomarkers, the biomarkers selected from two or more of AGR-2, midkine and CA125; two or more of CA125, IL-6, IL-8, CRP, SAA and SAP; two or more of IL-6, IL-8, CRP, SAA and SAP; and at least one of CA125, IL-6, IL-8, CRP, SAA and SAP and at least one of midkine or AGR-2 to determine levels of two or more biomarkers and then subjecting the levels to an algorithm generated from a first knowledge base of data comprising the levels of the same biomarkers from a subject of known status with respect to the condition wherein the algorithm provides an index of probability of the subject having or not having the condition.

In particular, the present invention provides a panel of ligands to biomarkers useful in the detection of a gynecological condition, the panel comprising ligands to two or more of AGR-2, midkine or CA125; two or more of CA125, IL-6, IL-8, CRP, SAA or SAP; two or more of IL-6, IL-8, CRP, SAA or SAP; or at least one of CA125, IL-6, IL-8, CRP, SAA or SAP and at least one of midkine or AGR-2.

In an alternative embodiment, the present invention contemplates a panel of biomarkers for the detection of a gynecological condition in a subject, the panel comprising agents which bind specifically to biomarkers, the biomarkers selected from two or more of AGR-2, midkine and CA125; two or more of CA125, IL-6, IL-8, CRP, SAA and SAP; two or more of IL-6, IL-8, CRP, SAA and SAP; and at least one of CA125, IL-6, IL-8, CRP, SAA and SAP and at least one of midkine or AGR-2 to determine levels of two or more biomarkers wherein an alteration in the levels of the biomarkers is indicative of the gynecological condition.

The combinations of biomarkers contemplated herein include from two biomarkers to nine biomarkers such as 2, 3, 4, 5, 6, 7, 8 or 9 biomarkers. The levels or concentrations of the biomarkers provide the input test data referred to herein as a “second knowledge base of data”. The second knowledge base of data either is considered relative to a control or is fed into an algorithm generated by a “first knowledge base of data” which comprise information of the levels of biomarkers in a subject with a known gynecological condition. The second knowledge base of data is from a subject of unknown status with respect to a gynecological condition. The output of the algorithm is a probability or risk factor, referred to herein as an index of probability, of a subject having a particular gynecological condition or not having the condition.

The two or more biomarkers include and comprise CA125, AGR-2; CA125, midkine; CA125, IL-6; CA125, IL-8; CA125, CRP; CA125, SAA; CA125, SAP; CA125; IL-6, IL-8; IL-6, CRP; IL-6, SAA; IL-6, SAP; IL-6; IL-6, midkine; IL-6, AGR-2; IL-8, CRP; IL-8, SAA; IL-8 SAP; IL-8; IL-8, midkine; IL-8, AGR-2; CRP, SAA; CRP, SAP; CRP; CRP, midkine; CRP, AGR-2; SAA, SAP; SAA; SAA, midkine; SAA, AGR-2; SAP; SAP, midkine; SAP, AGR-2; and midkine, AGR-2. Furthermore, the present invention extends to second knowledge base of data comprising the ratios of two or more markers such as ratios of CA125, IL-6; CA125, IL-8; CA125, CRP; CA125. SAA; CA125, SAP; CA125; CA125, midkine; CA125, AGR-2; IL-6, IL-8; IL-6, CRP; IL-6, SAA; IL-6, SAP; IL-6; IL-6, midkine; IL-6, AGR-2; IL-8, CRP; IL-8, SAA; IL-8 SAP; IL-8; IL-8, midkine; IL-8, AGR-2; CRP, SAA; CRP, SAP; CRP; CRP, midkine; CRP, AGR-2; SAA, SAP; SAA; SAA, midkine; SAA, AGR-2; SAP; SAP, midkine; SAP, AGR-2; and midkine, AGR-2.

In an alternative embodiment, a single biomarker is monitored in the form of AGR-2 or midkine. Furthermore, AGR-2 or midkine may be screened for in combination with one or more other markers. CA125 may also be measured in accordance with this aspect of the invention.

The agents which “specifically bind” to the biomarkers generally include an immunointeractive molecule such as an antibody or hybrid, derivative including a recombinant or modified form thereof or an antigen-binding fragment thereof. The agents may also be a receptor or other ligand. These agents assist in determining the level of the biomarkers. Information on the level is input data for the algorithm.

Hence, the present invention further provides a panel of immobilized ligands to two or more of AGR-2, midkine and/or CA125; two or more of CA125, IL-6, IL-8, CRP, SAA and/or SAP; two or more of IL-6, IL-8, CRP, SAA and/or SAP; or at least one of CA125, IL-6, IL-8, CRP, SAA and/or SAP and at least one of midkine and/or AGR-2.

Still another aspect of the present invention contemplates a kit for diagnosing the presence or absence of a gynecological condition, the kit comprising a composition of matter comprising the elements [X]ₙ, Y and [Z]ₘ wherein:

X is a ligand to a biomarker selected from CA125 and n is 0 or 1;
Y is a ligand to a biomarker selected from the list comprising, when n is 0, two or more of IL-6, IL-8, CRP, SAA and SAP or when n is 1, at least one of IL-6, IL-8, CRP, SAA and SAP; and
Z is a ligand to a biomarker selected from midkine and AGR-2 and m is 0 or 1;
the kit further comprising reagents to facilitate determination of the concentration of biomarker binding to a ligand. In use, the kit facilitates the determination of biomarkers. The levels am then compared to a control or subjected to an algorithm generated from a first knowledge base of data comprising the levels of the same biomarkers from a subject of known status with respect to the condition wherein the algorithm provides an index of probability of the subject having or not having the condition.

The kit may alternatively comprise reagents to detect the concentration of AGR-2 or midkine alone or in combination with CA125.

The present invention farther provides a panel of markers comprising the list [X]ₙ, [Y]ₓ and [Z]ₘ wherein:

### X is CA125 and n is 0 or 1;

Y is a marker selected from IL-6, IL-8, CRP, SAA and SAP provided that when n is 0, Y comprises two or more of the markers wherein x is 0 or 1; and
Z is two or more of AGR-2, midkine and/or CA125 and m is 0 or 1.

The ligands, such as antibodies specific to each of the biomarkers, enable the quantitative or qualitative detection or determination of the level of the at least two or more biomarkers. Reference to “level” includes concentration as weight per volume, activity per volume or units per volume or other convenient representative as well as ratios of levels.

The present invention further contemplates an assay for detecting ovarian cancer in a subject, the assay comprising contacting a sample from the subject with immobilized ligands to two or more of AGR-2, midkine and/or CA125; two or more of CA125, IL-6, IL-8, CRP, SAA and/or SAP; two or more of IL-6, IL-8, CRP, SAA and/or SAP; or at least one of CA125, IL-6, IL-8, SAA and/or SAP and at least one of midkine and/or AGR-2 for a time and under conditions sufficient for the biomarker to bind to a ligand and then detecting the level of binding which is indicative of the concentration of the biomarker wherein an alteration in the levels of the biomarkers is indicative of ovarian cancer.

In an alternative embodiment, the present invention is directed to an assay for detecting ovarian cancer in a subject, the assay comprising contacting a sample from the subject with immobilized ligands to two or more of AGR-2, midkine and/or CA125; two or more of CA125, IL-6, IL-8, CRP, SAA and/or SAP; two or more of IL-6, IL-8, CRP, SAA and/or SAP; or at least one of CA125, IL-6, IL-8, SAA and/or SAP and at least one of midkine and/or AGR-2 for a time and under conditions sufficient for the biomarker to bind to a ligand and then detecting the level of binding which is indicative of the concentration of the biomarker and subjecting the concentrations to an algorithm generated using levels of biomarkers in a subject having ovarian cancer to provide an index of probability that the subject has or does not have ovarian cancer.

In another alternative embodiment, the present invention provides an assay for detecting ovarian cancer in a subject, the assay comprising contacting a sample from the subject with an immobilized ligand to AGR-2 or midkine for a time and under conditions for AGR-2 or midkine to bind to its ligand which provides an indication of the concentration of AGR-2 or midkine or wherein an altered concentration of AGR-2 or midkine or is indicative of ovarian cancer. This aspect may also be combined with determining the concentration of CA125.

The “sample” is generally blood, plasma or serum, ascites, lymph fluid, tissue exudate, mucus, urine or respiratory fluid. Alternatively, the sample is a tissue sample which is being histologically examined.

By identifying levels of markers present in ovarian cancer patients and statistical methods useful in identifying which markers and groups of markers are useful in identifying ovarian cancer patients, a person of ordinary skill in the art, based on the disclosure herein, can identify panels that provide superior selectivity and sensitivity. Examples of panels providing discriminatory capability include, without limitation, biomarkers comprising CA125, AGR-2; CA125, midkine; CA125, IL-6; CA125, IL-8; CA125, CRP; CA125, SAA; CA125, SAP; CA125; CA125, midkine; CA125, AGR-2; IL-6, IL-8; IL-6, CRP; IL-6, SAA; IL-6, SAP; IL-6; IL-6, midkine; IL-6, AGR-2; IL-8, CRP; IL-8, SAA; IL-8 SAP; IL-8; IL-8, midkine; IL-8, AGR-2; CRP, SAA; CRP, SAP; CRP; CRP, midkine; CRP, AGR-2; SAA, SAP; SAA; SAA, midkine; SAA, AGR-2; SAP, midkine; SAP, AGR-2; and midkine, AGR-2. The panel may also comprise ligands to the aforementioned biomarkers.

The panel may also comprise AGR-2 alone or in combination with one or more other markers.

The panel may also comprise midkine alone or in combination with one or more other markers.

As indicated above, the “ligand” or “binding agent” and like terms, refers to any compound, composition or molecule capable of specifically or substantially specifically (that is with limited cross-reactivity) binding to an epitope on the biomarker. The “binding agent” generally has a single specificity. Notwithstanding, binding agents having multiple specificities for two or more biomarkers are also contemplated herein. The binding agents (or ligands) are typically antibodies, such as monoclonal antibodies, or derivatives or analogs thereof, but also include, without limitation: Fv fragments; single chain Fv (scFv) fragments; Fab′ fragments; F(ab′)2 fragments; humanized antibodies and antibody fragments; camelized antibodies and antibody fragments; and multivalent versions of the foregoing. Multivalent binding reagents also may be used, as appropriate, including without limitation: monospecific or bispecific antibodies; such as disulfide stabilized Fv fragments, scFv tandems [(scFv)₂ fragments], diabodies, tribodies or tetrabodies, which typically are covalently linked or otherwise stabilized (i.e. leucine zipper or helix stabilized) scFv fragments. “Binding agents” also include aptamers, as are described in the art.

Methods of making antigen-specific binding agents, including antibodies and their derivatives and analogs and aptamers, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163; 5,475,096; 5,840,867 and 6,544,776.

ECLIA, ELISA and Luminex LabMAP immunoassays are examples of suitable assays to detect levels of the biomarkers. In one example a first binding reagent/antibody is attached to a surface and a second binding reagent/antibody comprising a detectable group binds to the first antibody. Examples of detectable-groups include, for example and without limitation: fluorochromes, enzymes, epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays) or a non-planar surface, as with coated bead array technologies, where each “species” of bead is labeled with, for example, a fluorochrome (such as the Luminex technology described in U.S. Pat. Nos. 6,599,331, 6,592,822 and 6,268,222), or quantum dot technology (for example, as described in U.S. Pat. No. 6,306,610). Such assays may also be regarded as laboratory information management systems (LIMS).

In the bead-type immunoassays, the Luminex LabMAP system can be utilized. The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

As used herein, “immunoassay” refers to immune assays, typically, but not exclusively sandwich assays, capable of detecting and quantifying a desired biomarker, namely one of CA125, IL-6, IL-8, CRP, SAA, SAP, midkine and/or AGR-2.

Data generated from an assay to determine fluid or tissue levels of two, three or four or five or six or seven or eight or nine of the markers CA125, AGR-2, midkine, IL-6, IL-8, CRP, SAA and/or SAP, can be used to determine the likelihood of or progression of a gynecological condition in the subject. The input of data comprising the levels of two or more biomarkers is compared with a control or is put into the algorithm which provides a risk value of the likelihood that the subject has, for example, ovarian cancer. A treatment regime can also be monitored as well as a likelihood of a relapse.

In context of the present disclosure, “fluid” includes any blood fraction, for example serum or plasma, that can be analyzed according to the methods described herein. By measuring blood levels of a particular biomarker, it is meant that any appropriate blood fraction can be tested to determine blood levels and that data can be reported as a value present in that fraction. Other fluids contemplated herein include ascites, tissue exudate, urine, lymph fluid, mucus and respiratory fluid.

As described above, methods for diagnosing a gynecological condition by determining levels of specific identified biomarkers and using these levels as second knowledge base data in an algorithm generated with first knowledge base data or levels of the same biomarkers in patents with a known disease. Also provided are methods of detecting preclinical ovarian cancer comprising determining the presence and/or velocity of specific identified biomarkers in a subject's sample. By “velocity” it is meant the change in the concentration of the biomarker in a patient's sample over time.

As indicated above, a gynecological condition include cancer or a compilation thereof. The term “cancer” as used herein includes all cancers generally encompassed by a “gynecological cancer”. In one embodiment, a gynecological cancer, including, but not limited to, tubal metaplasia, ovarian serous borderline neoplasms, serous adenocarcinomas, low-grade mucinous neoplasms and endometrial tumors. In a specific embodiment, the gynecological cancer is an ovarian neoplasm, undergoing aberrant Mullerian epithelial differentiation. Other gynecological conditions contemplated herein include inflammatory disorders such as endometriosis.

The term “sample” as used herein means any sample containing cancer cells that one wishes to detect including, but not limited to, biological fluids (including blood, plasma, serum, ascites), tissue extracts, freshly harvested cells, and lysates of cells which have been incubated in cell cultures. In a particular embodiment, the sample is gynecological tissue, blood, serum, plasma or ascites.

As indicated above, the “subject” can be any mammal, generally human, suspected of having or having a gynecological condition. The subject may be referred to as a patient and is a female mammal suspected of having or having a gynecological condition or at risk of developing same. The term “condition” also includes complications arising therefrom.

The term “control sample” includes any sample that can be used to establish a first knowledge base of data from subjects with a known disease status.

The method of the subject invention may be used in the diagnosis and staging of a gynecological condition such as a gynecological cancer including ovarian cancer. The present invention may also be used to monitor the progression of a condition and to monitor whether a particular treatment is effective or not. In particular, the method can be used to confirm the absence or amelioration of the symptoms of the condition such as following surgery, chemotherapy, and/or radiation therapy. The methods can further be used to monitor chemotherapy and aberrant tissue reappearance.

In an embodiment, the subject invention contemplates a method for monitoring the progression of a gynecological condition in a patient, comprising:
(a) providing a sample from a patient;
(b) determining the level of two or more of AGR-2, midkine and/or CA125; two or more of CA125, IL-6, IL-8, CRP, SAA, SAP, midkine and/or AGR-2 biomarkers or AGR-2 or midkine alone and subjecting the levels to an algorithm to provide an index of probability of the patient having a gynecological condition; and
(c) repeating steps (a) and (b) at a later point in time and comparing the result of step (b) with the result of step (c) wherein a difference in the index of probability is indicative of the progression of the condition in the patient.

In an alternative, the subject invention contemplates a method for monitoring the progression of a gynecological condition in a patient, comprising:
(a) providing a sample from a patient;
(b) determining the level of two or more of AGR-2, midkine and/or CA125; two or more of CA125, IL-6, IL-8, CRP, SAA, SAP, midkine and/or AGR-2 biomarkers or AGR-2 or midkine alone and comparing the levels to a control wherein an alteration in the levels provides an index of probability of the patient having a gynecological condition; and
(c) repeating steps (a) and (b) at a later point in time and comparing the result of step (b) with the result of step (c) wherein a difference in the index of probability is indicative of the progression of the condition in the patient.

In particular, an increased index of probability of a disease condition at the later time point may indicate that the condition is progressing and that the treatment (if applicable) is not being effective. In contrast, a decreased index of probability at the later time point may indicate that the condition is regressing and that the treatment (if applicable) is effective.

In another embodiment of a method is provided for determining whether or not a gynecological cancer is benign in a patient comprising:
(a) providing a sample from the patient;
(b) detecting the level of two or more of AGR-2, midkine and/or CA125; CA125, IL-6, IL-8, CRP, SAA, SAP, midkine and/or AGR-2 biomarkers or AGR-2 or midkine alone and subjecting the levels to an algorithm to provide an index of probability of the patient having a gynecological cancer, and
(c) monitoring the indices of probability over time wherein a reduced index over time indicates that the cancer is benign.

In a further embodiment, a method is provided for determining whether or not a gynecological cancer is benign in a patient comprising:
(a) providing a sample from the patient;
(b) detecting the level of two or more of AGR-2, midkine and/or CA125; CA125, IL-6, IL-8, CRP, SAA, SAP, midkine and/or AGR-2 biomarkers or AGR-2, or midkine alone and comparing the levels to a control wherein an alteration in the levels provides an index of probability of the patient having a gynecological cancer; and
(c) monitoring the indices of probability over time wherein a reduced index over time indicates that the cancer is benign.

In an embodiment of the present invention, a method is provided for distinguishing between non-invasive and invasive gynecological cancers, comprising:
(a) providing a sample from a patient;
(b) determining the level of two or more of AGR-2, midkine and/or CA125; CA125, IL-6, 11-8, CRP, SAA, SAP, midkine and/or AGR-2 biomarkers or AGR-2 or midkine alone; and
(c) comparing the indices of probability over time and subjecting the levels to an algorithm to provide an index of probability of the patient having a gynecological condition wherein an increased index indicates that the cancer is invasive.

In a further embodiment of the present invention, a method is provided for distinguishing between non-invasive and invasive gynecological cancers, comprising:
(a) providing a sample from a patient;
(b) determining the level of two or more of AGR-2, midkine and/or CA125; CA125, IL-6, 11-8, CRP, SAA, SAP, midkine and/or AGR-2 biomarkers or AGR-2 or midkine alone; and
(c) comparing the indices of probability over time and comparing the levels to a control wherein an alteration in the levels provides an index of probability of the patient having a gynecological cancer.

In another embodiment, the invention contemplates a method for determining the potential risk to a patient of developing gynecological neoplasms, comprising:
(a) providing a sample from the patient;
(b) detecting the level of two or more AGR-2, midkine and/or CA125; CA125, IL-6, 11-8, CRP, SAA, SAP, midkine and/or AGR-2 biomarkers or AGR-2 or midkine alone and subjecting the levels to an algorithm to provide an index of probability of the patient having a gynecological condition; and
(c) comparing the indices of probability over time wherein a decreased index indicates that a patient is at a low risk of developing gynecological neoplasms.

In a further embodiment, the invention contemplates a method for determining the potential risk to a patient of developing gynecological neoplasms, comprising:
(a) providing a sample from the patient;
(b) detecting the level of two or more AGR-2, midkine and/or CA125; CA125, IL-6, 11-8, CRP, SAA, SAP, midkine and/or AGR-2 biomarkers or AGR-2 or midkine alone and comparing the levels to a control wherein an alteration in the levels provides an index of probability of the patient having a gynecological cancer, and
(c) comparing the indices of probability over time wherein a decreased index indicates that a patient is at a low risk of developing gynecological neoplasms.

In relation to determining the concentration of AGR-2 or midkine alone, an altered concentration (i.e. an increase or decrease) in one or more of AGR-2 or midkine is deemed to increase the index of probability of the presence of a disease condition. This aspect may also be in combination with determining the concentration of CA125.

As indicated above, antibodies may be used in any of a number of immunoassays which rely on the binding interaction between an antigenic determinant of the biomarker and the antibodies. Examples of such assays are radioimmunoassay, enzyme immunoassays (e.g. ECLIA, ELISA), immunofluorescence, immunoprecipitation, latex agglutination, hemagglutination and histochemical tests. The antibodies may be used to detect and quantify the level of the biomarker in a sample in order to determine its role in cancer and to diagnose the cancer.

In particular, the antibodies of the present invention may also be used in immunohistochemical analyses, for example, at the cellular and subcellular level, to detect a biomarker, to localize it to particular cells and tissues, and to specific subcellular locations, and to quantitate the level of expression.

Cytochemical techniques known in the art for localizing antigens using light and electron microscopy may be used to detect the biomarker. Generally, an antibody of the present invention may be labeled with a detectable substance and a biomarker protein may be localized in tissues and cells based upon the presence of the detectable substance. Examples of detectable substances include, but are not limited to, the following: radioisotopes (e.g. ³H, ¹⁴C ³⁵S, ¹²⁵, ¹³¹I), fluorescent labels (e.g. FITC, rhodamine, lanthanide phosphors), luminescent labels such as luminol; enzymatic labels (e.g. horseradish peroxidase, beta-galactosidase, luciferase, alkaline phosphatase, acetylcholinesterase), biotinyl groups (which can be detected by marked avidin e.g. streptavidin containing a fluorescent marker or enzymatic activity that can be detected by optical or calorimetric methods), predetermined polypeptide epitopes recognized by a secondary reporter (e.g leucine zipper pair sequences, binding sites for secondary antibodies, metal binding domains; epitope tags). In some embodiments, labels are attached via spacer arms of various lengths to reduce potential steric hindrance. Antibodies may also be coupled to electron dense substances, such as ferritin or colloidal gold, which are readily visualized by electron microscopy.

The antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip) Indirect methods may also be employed in which the primary antigen-antibody reaction is amplified by the introduction of a second antibody, having specificity for the antibody reactive against biomarker protein. By way of example, if the antibody having specificity against biomarker protein is a rabbit IgG antibody, the second antibody may be goat anti-rabbit gamma-globulin labeled with a detectable substance as described herein.

Where a radioactive label is used as a detectable substance, the biomarker may be localized by radioautography. The results of radioautography may be quantitated by determining the density of particles in the radioautographs by various optical methods, or by counting the grains.

Labeled antibodies against biomarker proteins may be used in locating tumor tissue in patients undergoing surgery i.e. in imaging. Typically for in vivo applications, antibodies are labeled with radioactive labels (e.g. iodine-123, iodine-125, iodine-131, gallium-67, technetium-99, and indium-111). Labeled antibody preparations may be administered to a patient intravenously in an appropriate carrier at a time several hours to four days before the tissue is imaged. During this period unbound fractions are cleared from the patient and the only remaining antibodies are those associated with tumor tissue. The presence of the isotope is detected using a suitable gamma camera. The labeled tissue can be correlated with known markers on the patient's body to pinpoint the location of the tumor for the surgeon.

Accordingly, in another embodiment the present invention provides a method for detecting cancer in a patient comprising:
(a) providing a sample from the patient;
(b) contacting the sample with an antibodies which bind to AGR-2, midkine, CA125, IL-6, IL-8, CRP, SAA and/or SAP biomarkers to determine the levels of two or more biomarkers or the levels of AGR-2 or midkine alone or in combination with CA125 and subjecting the levels to an algorithm to provide an index of probability of the patient having a gynecological condition; and
(c) diagnosing the risk of the patient having cancer based on the index of probability.

Alternatively, the present invention provides a method for detecting cancer in a patient comprising:
(a) providing a sample from the patient;
(b) contacting the sample with an antibodies which bind to AGR-2, midkine, CA125, IL-6, IL-8, CRP, SAA and/or SAP biomarkers to determine the levels of two or more biomarkers or the levels of AGR-2 or midkine alone or in combination with CA125 and comparing the levels to a control wherein an alteration in levels provides can index of probability of a patient having a gynecological condition; and
(c) diagnosing the risk of the patient having cancer based on the index of probability.

The methods of the present invention described herein may also be performed using microarrays, such as oligonucleotide arrays, cDNA arrays, genomic DNA arrays, or tissue arrays. Preferably the arrays are tissue microarrays.

In one embodiment, the method of the present invention involves the detection of expression of nucleic acid molecules encoding the biomarkers and to determine the level of biomarkers based on level of expression. Those skilled in the art can construct nucleotide probes for use in the detection of mRNA sequences encoding the biomarker in samples. Suitable probes include nucleic acid molecules based on nucleic acid sequences encoding at least five sequential amino acids from regions of the biomarker, preferably they comprise 15 to 30 nucleotides. A nucleotide probe may be labeled with a detectable substance such as a radioactive label which provides for an adequate signal and has sufficient half-life such as ³²P, ³H, ⁴⁴C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, Molecular Cloning. A Laboratory Manual. (2nd ed), 1989. The nucleic acid probes may be used to detect genes, preferably in human cells, that encode the biomarker. The nucleotide probes may also be useful in the diagnosis of disorders involving a biomarker, in monitoring the progression of such disorders, or in monitoring a therapeutic treatment. In an embodiment, the probes are used in the diagnosis of, and in monitoring the progression of a gynecological cancer such as ovarian cancer.

The probe may be used in hybridization techniques to detect expression of genes that encode biomarker proteins. The technique generally involves contacting and incubating nucleic acids (e.g. mRNA) obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe if any are detected.

The detection of mRNA may involve converting the mRNA to cDNA and/or the amplification of specific gene sequences using an amplification method such as polymerase chain reaction (PCR), followed by the analysis of the amplified molecules using techniques known to those skilled in the art. Suitable primers can be routinely designed by one of skill in the art.

Hybridization and amplification techniques described herein may be used to assay qualitative and quantitative aspects of expression of genes encoding the biomarker. For example, RNA may be isolated from a cell type or tissue known to express a gene encoding the biomarker, and tested utilizing the hybridization (e.g. standard Northern analyses) or PCR techniques referred to herein. The techniques may be used to detect differences in transcript size which may be due to normal or abnormal alternative splicing. The techniques may be used to detect quantitative differences between levels of full length and/or alternatively splice transcripts detected in normal individuals relative to those individuals exhibiting symptoms of a cancer involving a biomarker protein or gene.

The primers and probes may be used in the above described methods in situ i.e. directly on tissue sections (fixed and/or frozen) of patient tissue obtained from biopsies or resections.

Accordingly, the present invention provides a method of detecting cancer in a patient comprising:
(a) providing a sample from the patient;
(b) extracting nucleic acid molecules comprising mRNA from a biomarker gene or portion thereof from the sample;
(c) amplifying the extracted mRNA using the polymerase chain reaction;
(d) determining the level of mRNA encoding the biomarker, and
(e) subjecting the levels of two or more biomarkers to an algorithm which provides an index of probability of the patient having cancer.

The biomarker mRNA is selected from mRNA encoding two or more of AGR-2, midkine, CA125, IL-6, IL-8, CRP, SAA and/or SAP.

The methods described herein may be performed by utilizing pre-packaged diagnostic kits comprising the necessary reagents to perform any of the methods of the invention. For example, the kits may include at least one specific nucleic acid or antibody described herein, which may be conveniently used, e.g. in clinical settings, to screen and diagnose patients and to screen and identify those individuals exhibiting a predisposition to developing cancer. The kits may also include nucleic acid primers for amplifying nucleic, acids encoding the biomarker in the polymerase chain reaction. The kits can also include nucleotides, enzymes and buffers useful in the method of the invention as well as electrophoretic markers such as a 200 bp ladder. The kit also includes detailed instructions for carrying out the methods of the present invention.

The present invention further provides an algorithm-based screening assay to screen samples from patients. Generally, input data are collected based on levels of two or more biomarkers (or levels of expression of genes encoding two or more biomarkers) and subjected to an algorithm to assess the statistical significance of any elevation or reduction in levels which information is then output data. Computer software and hardware for assessing input data are encompassed by the present invention.

Another aspect of the present invention contemplates a method of treating a patient with a gynecological condition such as ovarian cancer the method comprising subjecting the patient to a diagnostic assay to determine an index of probability of the patient having the condition, the biomarkers selected from two or more of AGR-2, midkine, and/or CA125; two or more of CA125, IL-6, IL-8, CRP, SAA and/or SAP; two or more of IL-6, IL-8, CRP, SAA and/or SAP; or at least one of CA125, IL-6, IL-8, CRP, SAA and/or SAP and at least one of midkine and/or AGR-2; and where there is a risk of the patient having the condition, subjecting the patient to surgical ablation, chemotherapy and/or radiotherapy; and then monitoring index of probability over time.

The second detected biomarkers may be the same or different to the first detected biomarkers.

The present invention further provides the use the levels of two or more biomarkers selected from CA125, IL-6, IL-8, CRP, SAA and SAP in the generation of an index of probability for use in a diagnostic assay to detect ovarian cancer in a subject.

Another aspect of the present invention provides use the levels of two or more biomarkers selected from CA125, IL-6, IL-8, CRP, SAA and SAP in the generation of an algorithm for use in a diagnostic assay to detect ovarian cancer in a subject.

Still another aspect of the present invention provides the use of levels of AGR-2 in the generation of an assay to detect ovarian cancer or other gynecological condition in a subject.

The assay of the present invention permits integration into existing or newly developed pathology architecture or platform systems. For example, the present invention contemplates a method of allowing a user to determine the status of a subject with respect to a gynecological cancer or subtype thereof or stage of cancer, the method including:
(a) receiving data in the form of levels or concentrations of CA125 and one or more of AGR-2, midkine, CRP, L-6, IL-8, SAA and SAP from the user via a communications network;
(b) processing the subject data via an algorithm which provides a disease index value;
(c) determining the status of the subject in accordance with the results of the disease index value in comparison with predetermined values; and
(d) transferring an indication of the status of the subject to the user via the communications network.

Conveniently, the method generally further includes:
(a) having the user determine the data using a remote end station; and
(b) transferring the data from the end station to the base station via the communications network.

The base station can include first and second processing systems, in which case the method can include:
(a) transferring the data to the first processing system;
(b) transferring the data to the second processing system; and
(c) causing the first processing system to perform the algorithmic function to generate the disease index value.

The method may also include:
(a) transferring the results of the algorithmic function to the first processing system; and
(b) causing the first processing system to determine the status of the subject.

In this case, the method also includes at lest one of:
(a) transferring the data between the communications network and the first processing system through a first firewall; and
(b) transferring the data between the first and the second processing systems through a second firewall.

The second processing system may be coupled to a database adapted to store predetermined data and/or the algorithm, the method include:
(a) querying the database to obtain at least selected predetermined data or access to the algorithm from the database; and
(b) comparing the selected predetermined data to the subject data or generating a predicted probability index.

The second processing system can be coupled to a database, the method including storing the data in the database.

The method can also include having the user determine the data using a secure array, the secure array of elements capable of determining the level of biomarker and having a number of features each located at respective position(s) on the respective code. In this case, the method typically includes causing the base station to:
(a) determine the code from the data;
(b) determine a layout indicating the position of each feature on the array; and
(c) determine the parameter values in accordance with the determined layout, and the data.

The method can also include causing the base station to:
(a) determine payment information, the payment information representing the provision of payment by the user; and
(b) perform the comparison in response to the determination of the payment information.

The present invention also provides a base station for determining the status of a subject with respect to a gynecological cancer or a subtype thereof or a stage of the cancer, the base station including:
(a) a store method;
(b) a processing system, the processing system being adapted to:
   (i) receive subject data from the user via a communications network, the data including levels or concentrations of two or more biomarkers selected from AGR-2, midkine, CA125, IL-6, IL-8, CRP, SAA and SAP from a subject;
   (ii) performing an algorithmic function including comparing the data to predetermined data;
   (iii) determining the status of the subject in accordance with the results of the algorithmic function including the comparison; and
(c) output an indication of the status of the subject to the user via the communications network.

The processing system can be adapted to receive data from a remote end station adapted to determine the data.

The processing system may include:
(a) a first processing system adapted to:
   (i) receive the data; and
   (ii) determine the status of the subject in accordance with the results of the algorithmic function including comparing the data; and
(b) a second processing system adapted to:
   (i) receive the data from the processing system;
   (ii) perform the algorithmic function including the comparison; and
   (iii) transfer the results to the first processing system.

The base station typically includes:
(a) a first firewall for coupling the first processing system to the communications network; and
(b) a second firewall for coupling the first and the second processing systems.

The processing system can be coupled to a database, the processing system being adapted to store the data in the database.

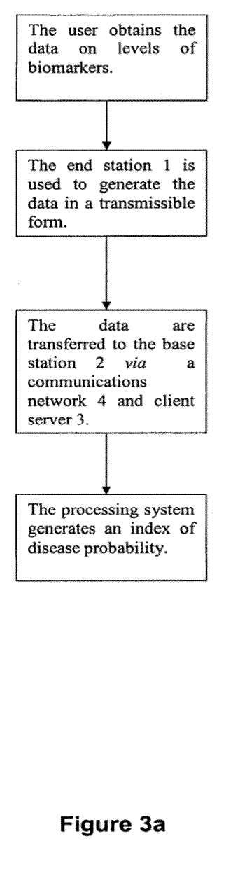

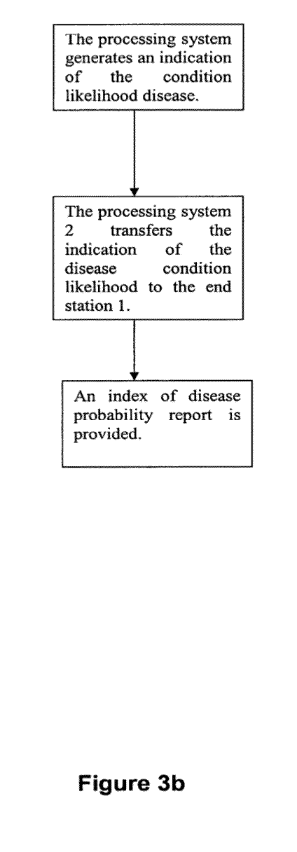

Reference to an “algorithm” or “algorithmic functions” as outlined above includes the performance of a multivariate analysis function. A range of different architectures and platforms may be implemented in addition to those described above. It will be appreciated that any form of architecture suitable for implementing the present invention may be used. However, one beneficial technique is the use of distributed architectures. In particular, a number of end stations 1 (FIG. 3) may be provided at respective geographical locations. This can increase the efficiency of the system by reducing data bandwidth costs and requirements, as well as ensuring that if one base station becomes congested or a fault occurs, other end stations 1 could take over. This also allows load sharing or the like, to ensure access to the system is available at all times.

In this case, it would be necessary to ensure that the base station 2 contains the same information and signature such that different end stations 1 can be used.

It will also be appreciated that in one example, the end stations 1 can be hand-held devices, such as PDAs, mobile phones, or the like, which are capable of transferring the subject data to the base station via a communications network 4 such as the Internet, and receiving the reports.

In the above aspects, the term “data” means the levels or concentrations of the biomarkers. The “communications network” includes the internet. When a server is used, it is generally a client server or more particularly a simple object application protocol (SOAP).

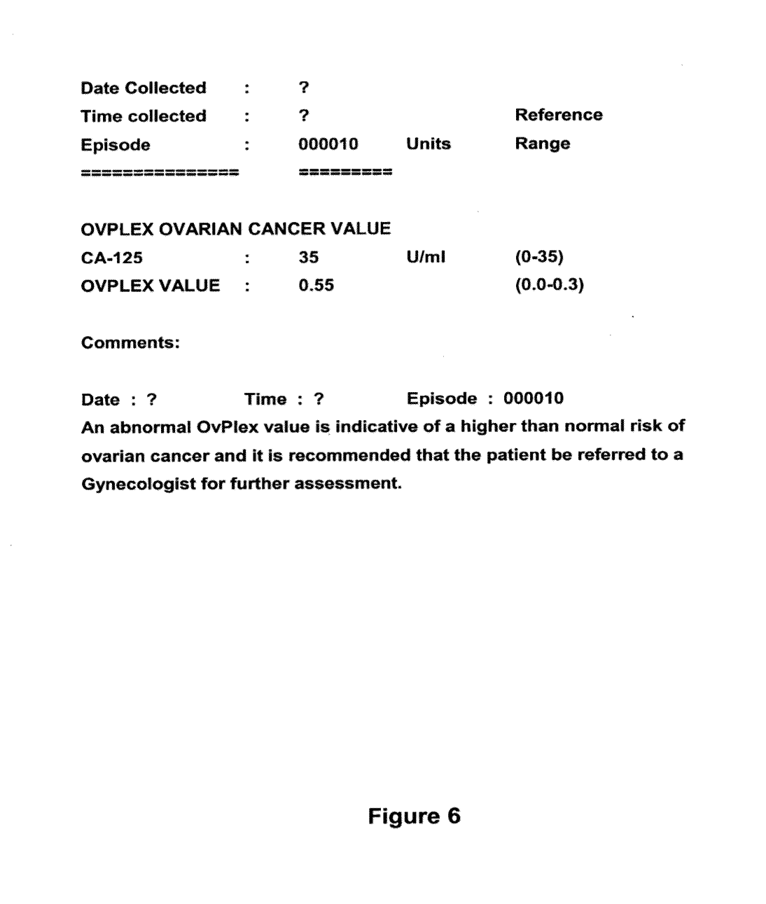

A report outlining the likelihood of gynecological cancer by the subject is issued. An example of such a report is provided in FIG. 6.

The present invention is further described by the following non-limiting Examples. Materials and Methods relevant to these Examples are provided below.

Multiplex ELISA assays for IL-6 and IL-8 assay was obtained from Biorad. Cardiovascular Panel 2 assay (CVD2) to measure Serum Amyloid A, Serum Amyloid P and C-reactive protein was obtained from Lincoplex. Additionally, CA 125 assays were performed on all samples using Roche assay kit performed on a Roche analyzer platform. The Roche assay is an electrochemiluminesence immunoassay “ECLIA”, where a biomarker/two labeled antibody sandwich is coupled to microparticles. The microparticles are magnetically captured onto the surface of the electrode. Application of a voltage to the electrode induces a chemiluminescent emission which is measured by a photomultiplier.

Both midkine and AGR2 were measured by standard sandwich ELISA techniques in a conventional 96 well plate format.

Immunohistochemical localization of immunoreactive (ir)-AGR-2 was performed using affinity purified rabbit anti-AGR-2 antibody (Liu et al, Cancer Res 65 (9):3796-3805, 2005). The antibody was diluted (1:500) in Tris-buffered saline containing 0.5% v/v Tween-20 and 3% w/v skim milk powder and incubated with rehydrated paraffin sections for two hours at room temperature. The sections were then incubated with a biotin-linked anti-rabbit IgG followed by incubation with streptavidin-HRP reagent and ir-AGR-2 was visualized using diaminobenzidine as chromogen. Sections were counterstained with haematoxylin prior to visual examination.

Plasma samples from women with diagnosed ovarian cancer were obtained from various hospitals or clinics denoted source I through IV. Control plasma samples from healthy individuals were obtained from the same sources. All samples when received were stored frozen at ˜80C until processed. Additional control plasma samples from women diagnosed with endometriosis were also obtained.

### Example 1

### Selection of Biomarkers

The following biomarkers were selected for inclusion in a panel, with or without CA125: IL-6, IL-8, CRP, SAA and SAP. Additional biomarkers included midkine and AGR-2.

### Example 2

### Determination of Index of Probability

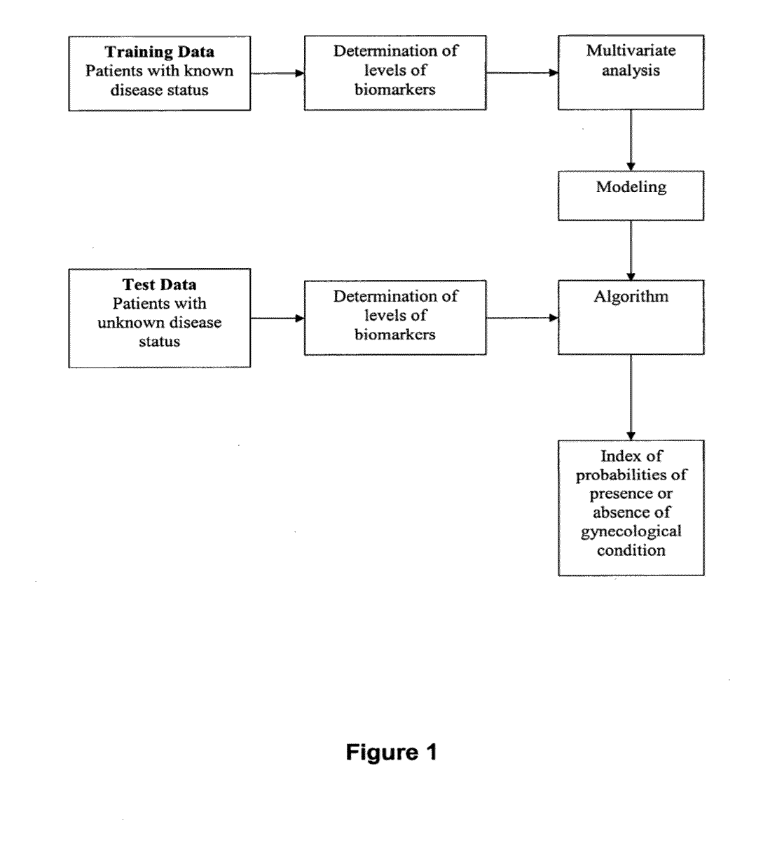

FIG. 1 provides a diagrammatic representation of the modeling leading to the algorithm used in the diagnostic assay. Training data in the form of the concentration of biomarkers from patients of known disease status are subjected to multivariate analysis to generate an algorithm. In essence, the assay is a diagnostic rule based on the application of a statistical and machine learning algorithm. Such an algorithm uses the relationships between biomarkers and disease status observed in training data (with known disease status) to infer relationships which are then used to predict the status of patients with unknown status. Practitioners skilled in the art of data analysis recognize that many different forms of inferring relationships in the training data may be used without materially changing the present invention.

The biomarker concentrations (i.e. levels) of two or more of the biomarkers in the training data enable the generation of an algorithm which provides a measurable relationship between biomarker levels and disease status in patients. In addition to “level” of biomarker, the present invention extends to ratios of two or more markers as input data for multivariate analysis leading to the algorithm.

Test data in the form of concentrations of biomarkers from patients of unknown status are then inserted into the algorithm and an index of probability is provided whether or not the patient has a gynecological condition.

### Example 3

### Development of Assay

A CA125 assay was performed using Roche CA125 II kit and performed using Roche E170 module analyser. A cut-off of value of 35 U/ml was employed.

Based on product insert data the performance levels expected of the CA125 assay are shown in Table 1.

TABLE 1

| Cut-off Value (U/mL) | Sensitivity | Specificity |
|---|---|---|
| 65 | 79% | 82% |
| 150* | 69% | 93% |
| 190 | 63% | 95% |

*Level of optimal clinical value (as defined in Roche CA125 II kit).

The biomarker panel assays were performed using multiplex bead assays, on a Biorad Bioplex 100 instrument. Samples included serous (64%), mucinous (7%), endometrioid (10%) and mullerian (4%) types.

Based on pathology the cancer sample bank contained Stage I to IV ovarian cancers.

Statistical analysis was performed to compare sensitivity and specificity of the conventional CA 125 assay and the biomarkers assay.

This analysis used a randomly selected set of samples to generate an algorithm model. The performance of the generated model was validated by prediction of a second independent sample set. This provides sensitivity and specificity for both model and validation sample sets. ROC curve analysis was conducted to compare statistical significance between the biomarkers and CA125 results.

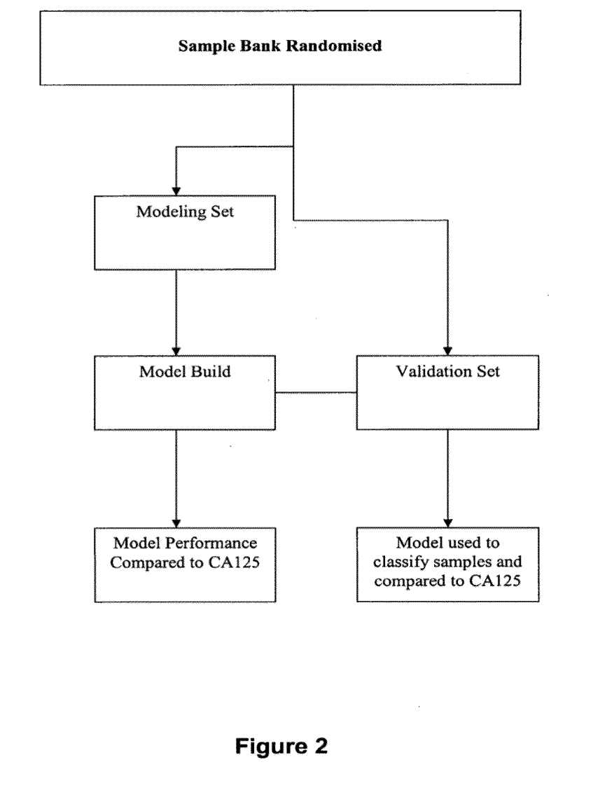

The model build and validation strategy is shown in FIG. 2. Results are shown in Table 2.

TABLE 2

| All Stages | CA125 | Biomarkers |
|---|---|---|
| Diagnostic Efficiency | 90.70% | 94.00% |
| AUC | 0.960 | 0.982* |
| Bootstrap Limits | 0.924-0.988 | 0.966-0.994 |
| Sensitivity | 92.6% | 91.2% |
| Specificity | 89.6% | 95.7% |

*Statistically signiticant at the 5% level (tail area probability = 0.012)

Stage I and II ovarian cancers were then compared and the results shown in Table 3.

TABLE 3

| Stage I and II only | CA125 | Biomarkers |
|---|---|---|
| Diagnostic Efficiency | 89.50% | 92.8% |
| AUC | 0.933 | 0.984 |
| Sensitivity | 89.20% | 89.2% |
| Specificity | 89.60% | 93.9% |

A comparison of all cancers is shown in Table 4.

TABLE 4

| All Cancer | CA125 | Biomarkers |
|---|---|---|
| Diagnostic Efficiency | 92.0% | 95.3% |
| AUC | 0.951 | 0.988 |
| Sensitivity | 91.4% | 92.1% |
| Specificity | 92.5% | 97.6% |

The analysis verified a higher level of performance of the biomarker assay compared to a conventional CA125 assay. This elevated performance level is present when considering either all ovarian cancers or only those classified as early stage (Stage I and II).

### Example 4

### Diagnostic Assay

Samples comprising plasma were allowed to thaw on ice, vortexed for 30 seconds then centrifuged for 5 minutes at 14,000 g. Dilutions of the plasma were then made from 1:3 to 1:40,000 in assay buffer.

In total 149 ovarian cancer samples, 212 control samples (includes 57 endometriosis samples) were submitted for testing. Ovarian cancers were classified by conventional means, as to their stage of disease progression. For analysis purposes all stage I and stage II samples have been denoted as Early stage and stage III and IV samples as Late stage disease.

The Stage breakdown for the entire ovarian cancer set is shown in Table 5.

TABLE 5

| | Stage I | Stage II | Stage III | Stage IV |
|---|---|---|---|---|
| Number of | 28 | 62 | 46 | 8 |
| samples | | | | |

The disease type diagnosis for the sample set is contained in Table 6.

TABLE 6

| | Serous | Clear Cell | Mucinous | Other |
|---|---|---|---|---|
| Number of | 97 | 12 | 11 | 29 |
| Samples | | | | |

ROC curves were generated for the individual analytes which demonstrates their individual diagnostic performance in detecting ovarian cancer. The results are shown in Table 7

TABLE 7

| Analyte Marker | ROC Plot Area Under Curve |
|---|---|
| CA125 | 0.9600 |
| CRP | 0.8491 |
| SAA | 0.7887 |
| IL-6 | 0.7089 |
| SAP | 0.5810 |
| IL-8 | 0.6954 |

Furthermore, it was identified that a ratio of CRP and SAP may produce improved performance over that of the individual markers alone. This ratio relates the concentration of SAP relative to CRP to disease state, where previously there had no evidence that SAP concentration may relate to ovarian cancer.

### Example 5

### Modeling

Initial analysis used weka software to assess various combinations of markers for their discrimination of all disease and control samples. This analysis was performed by splitting the data sets into two randomly picked sets. One set was then used as a modeling set to build a model, while the second data set acted as a validation group to determine the performance of the model with independent data. Additional analysis examined identification of early stage (stage I and II) subjects, by including only early stage subjects and controls within the validation group. In all cases, the performance of the marker set was assessed relative to the performance of the CA125 assay alone.

The best performing marker combinations were then independently analyzed using a logitboost algorithm model. Results of this analysis are detailed below.

Analysis of marker combinations with “All Stage Cancer” and with “Early Stage Cancer” is summarized in Table 8 below. Three combinations of markers were tested, the results for the validation set, and for combined model and validation (denoted “All Data”) is presented for comparison with CA125 alone. It can be seen that for all three models the area under the curve for the ROC plots is greater than that of CA125 alone, indicative of greater diagnostic utility. Analysis of the ROC curves found that in all but one data set, this increased diagnostic utility was statistically significant.

TABLE 8

| | Validation Set | | All Data |
|---|---|---|---|
| | All Stages | Early Stage | (model + validation sets) |
| CA125 alone | 0.960 | 0.933 | 0.950 |
| CA125 + CRP + SAA + IL- | 0.984 | 0.978 | 0.972 |
| 6 + IL-8 | | | |
| Statistical Significance | Yes at 1% level | Yes at 5% level | Yes at 0.1% level |
| CA125 + IL-6 + IL-8 + ratio | 0.984 | 0.976 | 0.971 |
| CRP:SAP + ratio | | | |
| SAA + SAP | | | |
| Statistical Significance | Yes at 5% level | Yes at 5% level | Yes at 1% level |
| CA125 + ratio CRP:SAP + | 0.977 | 0.946 | 0.966 |
| ratio SAA:SAP | | | |
| Statistical Significance | Yes at 5% level | No | Yes at 5% level |

In the above table, “CRP:SAP” means CRP is divided by SAP; “SAA:SAP” means SAA is divided by SAP.

It has been demonstrated that improvement on the diagnostic efficiency of CA125 has been achieved, the conventional “gold standard” diagnostic assay for ovarian cancer, by combining it with other markers.

Three combinations of markers with improved performance over CA125 in diagnosing not only all stage ovarian cancer, but that two of these marker combinations are statistically better than CA125 in detecting early stage disease, a factor vital to patient survival.

One marker included in these analysis is SAP in ratio combinations with two acute phase inflammation markers (CRP and SAA) can be utilized. Previously, SAP or its ratio with other markers had not been linked to ovarian cancer.

### Example 6

### Integration of the Assay into a Pathology Platform

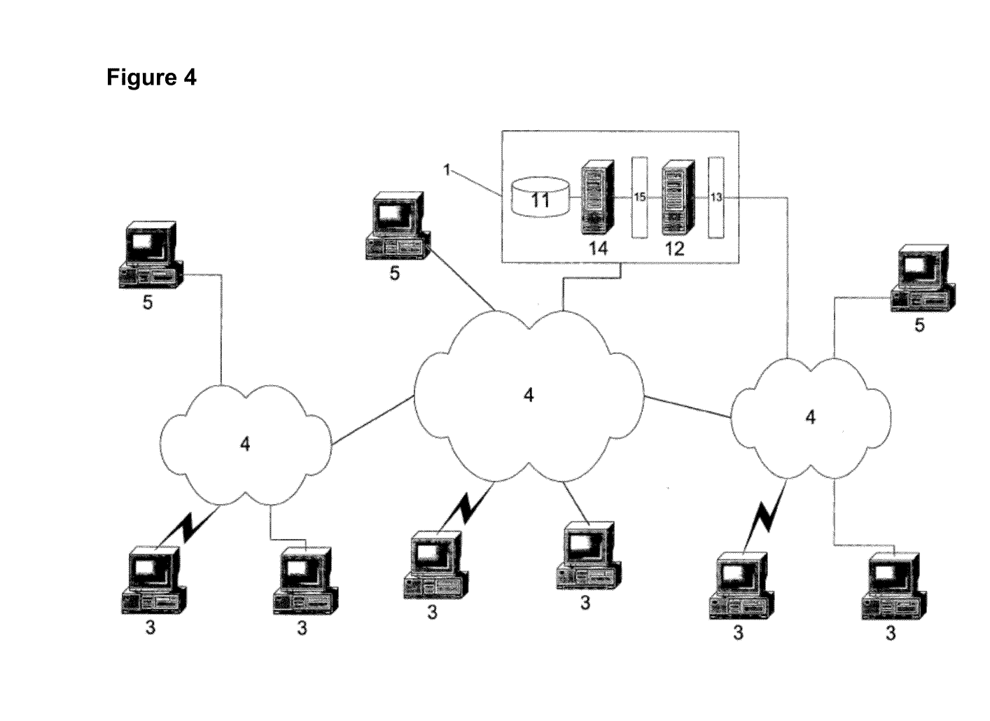

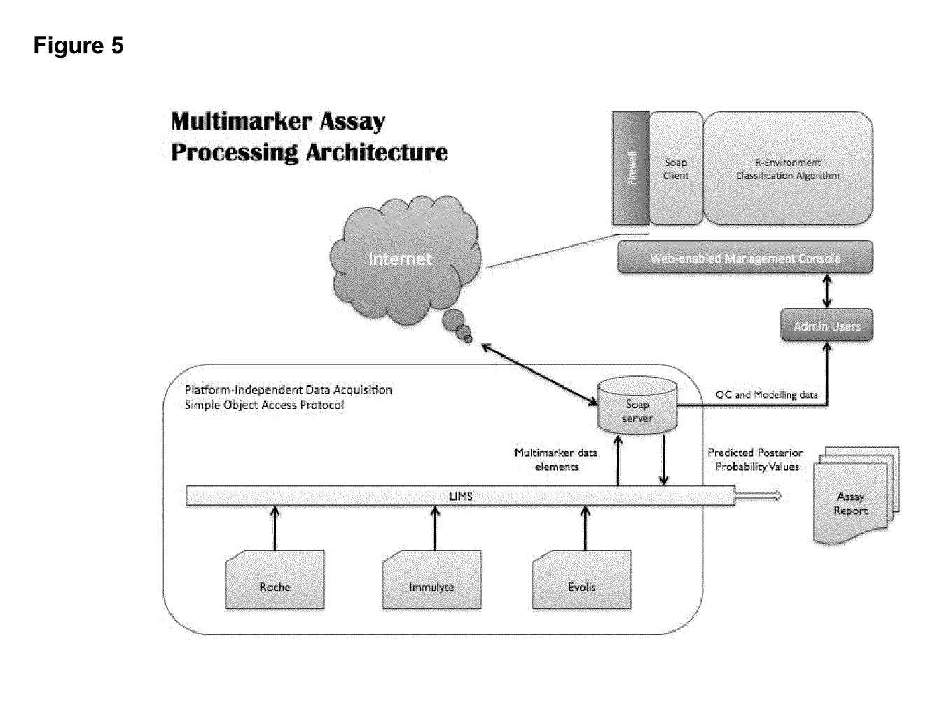

The levels or concentrations of combinations of biomarkers enables the generation of a predicted posterior probability value, i.e. likelihood that a sample came from a woman with ovarian cancer. The levels or concentrations of the biomarkers ultimately provides an index of probability for a patient sample of that sample being derived from a subject with or without ovarian cancer. The multimarker diagnostic assay is designed to be fully complementary with various pathology platforms used to determine the levels or concentrations of the biomarkers. Such platforms may be referred to as laboratory information management systems (LIMS). The level or concentration data of the biomarkers is conveniently transferred to a centralized processing serve to generate a predicted probability index via a multivariate classification algorithm. A report is generated to indicate the likelihood of ovarian cancer to the clinician. FIG. 6 provides an example of the report. FIGS. 3a and b and FIGS. 4 and 5 provide schematic representations of integration of the assay into a LIMS. The server is generally a client server such as a simple object application protocol (SOAP).

In relation to FIGS. 3a and b, the user obtains data on the levels or concentrations of the biomarkers. Two or more of AGR-2, midkine, CA125, IL-6, IL-8, CRP, SAA and SAP are selected. End station 1 generates data in a transmissible form. The data are transferred to base station 2 via a communications network 4 and client serves (e.g. SOAP) 3.

The processing system then generates an index of probability and an indication of the likelihood of the presence or absence of a disease condition. This information is then transferred to the end station 1. A report is then issued (see for example, FIG. 6). The scheme is represented in FIGS. 4 and 5.

### Example 7

### Anterior Gradient-2 (AGR-2)

Anterior gradient 2 (AGR-2) is the human homolog of the cement-gland gene XAG-2 that was previously described in Xenopus laevis (Aberger et al, Mech Dev 72(1-2):115-130, 1998) where this gene has been shown to be a crucial factor involved in cellular differentiation and development. In several human breast cancer cell lines, mRNA transcripts for AGR-2 have been shown to be coexpressed with oestrogen receptor (ER) suggesting that AGR-2 may play a role in the differentiation of hormonally responsive breast cancers (Thompson and Weigel, Biochem Biophys Res Commun 251(1):111-116, 1998).

Although the AGR-2 gene contains a signal sequence suggestive of protein secretion and the XAG-2 homolog has been shown to be secreted when expressed in Xenopus oocytes (Aberger et al, 1998 supra), there is currently no evidence to suggest that AGR-2 is secreted into the circulation in normal humans or in human cancer patients.

Using a rabbit polyclonal antiserum raised against human AGR-2 (Liu et al, 2005 supra) it was shown by immunohistochemical staining that immunoreactive (ir)-AGR-2 is totally absent in the epithelial cells of normal human ovary whereas the ovarian epithelium of ovarian carcinoma patients demonstrates distinct cytoplasmic, granular ir-AGR-2 staining of varying intensity. In all normal ovarian tissue examined (n=5), no ir-AGR-2 was detected in surface epithelium, however, occasional cells lining inclusion cysts demonstrated positive staining for ir-AGR-2. A series of five ovarian samples containing benign cysts (two mucinous and three serous) were examined and the mucinous cysts in particular showed strong ir-AGR-2 staining of virtually all columnar epithelium. Weaker ir-AGR-2 staining was observed in scattered differentiated epithelium of the serous benign cysts. In borderline serous ovarian tumors (n=5), approximately 50% of surface epithelium was generally immunostained for AGR-2 and this staining was primarily seen within complex glandular areas of the tumors. Four out of five grade 1 endometrioid tumors displayed strong ir-AGR-2 staining in the majority of epithelial cells, while the fifth case demonstrated ir-AGR-2 staining that was confined to approximately 10% of the epithelium. In three cases of grade 2 serous ovarian carcinoma displaying relatively poor cellular differentiation and little glandular formation, ir-AGR-2 was detected in scattered cells, predominantly within the more differentiated areas. Two additional grade 2 serous tumors of a more differentiated papillary type appeared to display greater ir-AGR-2 immunostaining, with more than 50% of the epithelium staining positive. Of four grade 3 serous tumors examined, one tumor demonstrated no ir-AGR-2 staining, while the remainder displayed distinct ir-AGR-2 in scattered cells, predominantly throughout the more differentiated regions of the tumor. An additional grade 3 clear cell carcinoma was shown to display strong ir-AGR-2 staining that was present in a far greater proportion of cells than the corresponding grade 3 serous tumors.

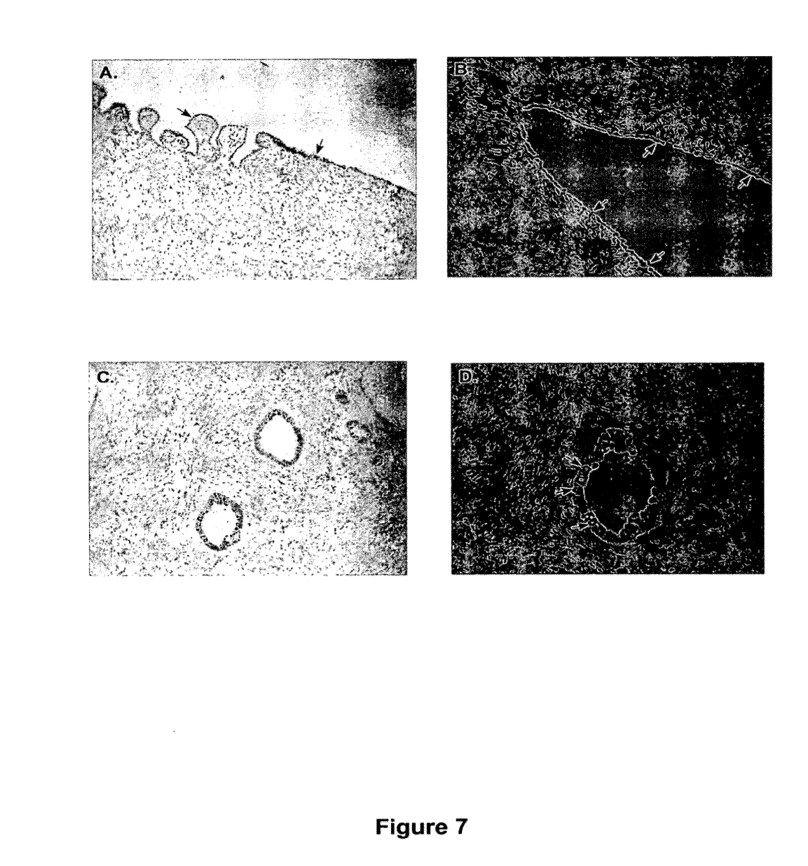

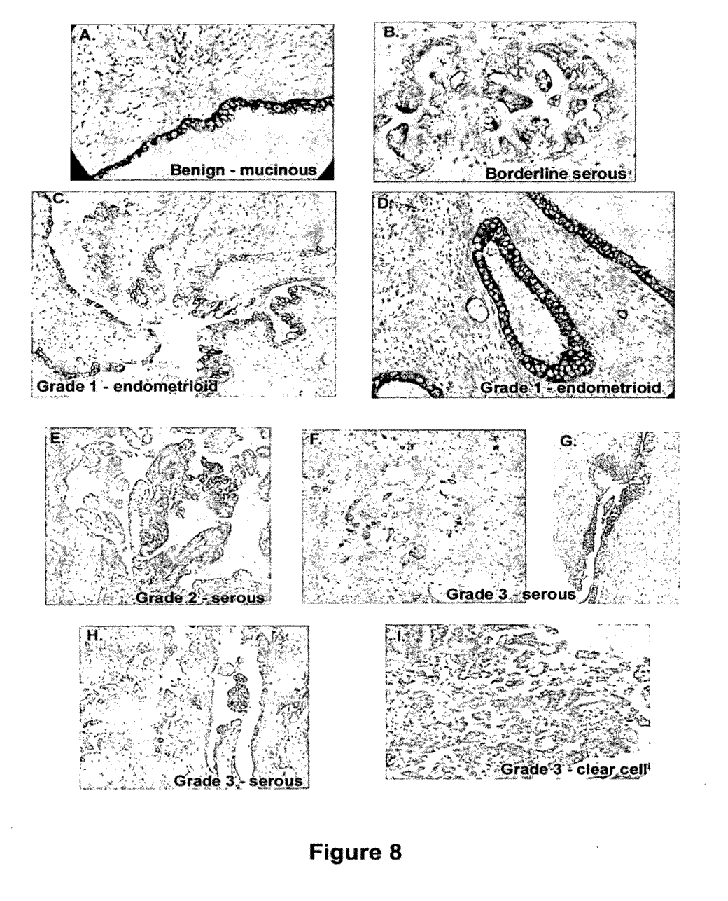

Overall, the immunostaining of epithelial-derived ovarian carcinoma of various types and grades demonstrates that ir-AGR-2 can be detected in virtually 100% of ovarian carcinoma tissue, but is absent in the epithelium of normal human ovary. Moreover, the prominent ir-AGR-2 staining detected in mucinous, endometrioid and clear cell as well as serous ovarian epithelial tumors suggests that AGR-2 may serve as a useful biomarker that can define multiple types of epithelial ovarian tumors. Furthermore, the present data suggest that although ir-AGR-2 can be demonstrated in ovarian tumors of varying grade, immunostaining appears to be more widespread in low grade tumors displaying more highly differentiated cells. The results are shown in FIGS. 7 to 8.

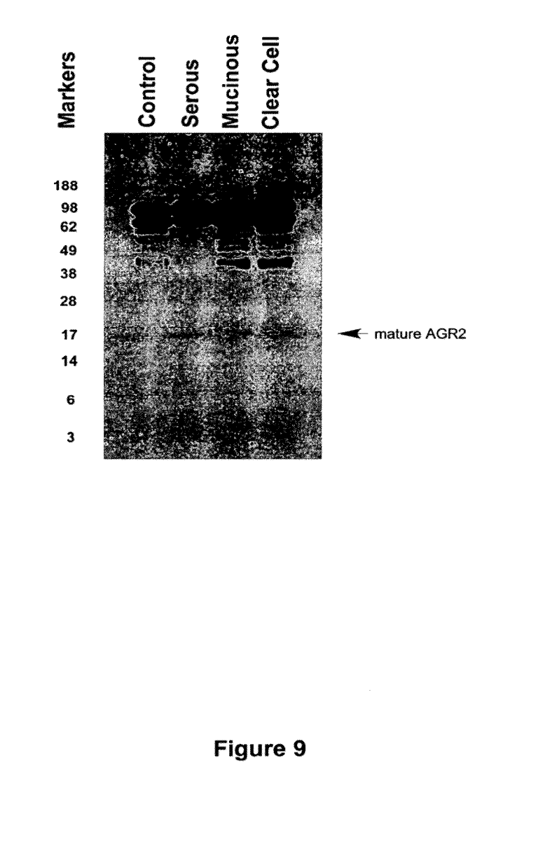

Studies demonstrated the presence of putative ir-AGR-2 species circulating in the plasma of a subset of ovarian cancer patients (FIG. 9). Individual patient plasma was obtained from control, serous, mucinous and clear cell ovarian cancer patients (3-6 per group) and pooled. The pooled plasma samples were then subjected to affinity depletion of the top six plasma proteins using an Agilent Multiple Affinity Removal System to concentrate the remaining plasma proteins and enhance the probability of detecting low abundance proteins such as AGR-2. The equivalent of 12 μg of depleted plasma proteins from each pool were Western blotted using rabbit anti-AGR-2 and visualized by chemiluminesence detection as described by Lieu et al, 2005 supra. Plasma obtained from mucinous and clear cell ovarian cancer patients demonstrated a weak immunoreactive species of approximately 18 kDa, consistent with the mass of mature AGR-2, while control subjects and plasma obtained from serous ovarian cancer patients showed no detectable ir-AGR-2 (FIG. 9). Additional immunoreactive species of higher apparent molecular mass also appeared to be expressed in a differential and tumour specific manner.

Collectively, these data indicate that ir-AGR-2 is produced by ovarian tumors and is secreted into the circulation. The differences in tissue expression and in the level of detectable ir-AGR-2 suggests that AGR-2 is differentially expressed and secreted by different ovarian tumor types. Notwithstanding, it is proposed that any alteration, i.e. an increase or decrease in ir-AGR-2 concentration is indicative of a gynecological condition.

### Example 8

### Using Markers CA125, Serum Amyloid-A, IL-8 and Midkine

Plasma samples were obtained from individuals with only stage I, II and III level disease. All patients with level IV disease were omitted as were those whose stage data were not available. Age matched controls were also assayed.

All patients and controls were randomly assigned to either modeling or validation data subsets, for the purpose of biomarker panel analysis.

The model set contained 74 disease and 96 controls. Of these 7 disease samples were negative by CA125 testing, having values lower than 35 U/ml. Of the controls 4 were given false positive results (e.g. values >/=35 U/ml) in CA 25 testing.

Using logitboost modeling in weka software, a model was built. In this model only 1 control sample was given a false positive result, and 3 disease samples falsely assigned as negative for ovarian cancer (Table 9).

TABLE 9

| Diagnostic | False | False | True | True | | | Diagnostic |
|---|---|---|---|---|---|---|---|
| Test | negatives | positives | negatives | positives | Sensitivity | Specificity | Efficiency |
| CA125 | 7 | 4 | 92 | 67 | 90.5% | 95.8% | 93.15% |
| CA125/SAA/ | 3 | 1 | 95 | 71 | 95.9% | 99.0% | 97.45% |
| IL8/MK | | | | | | | |

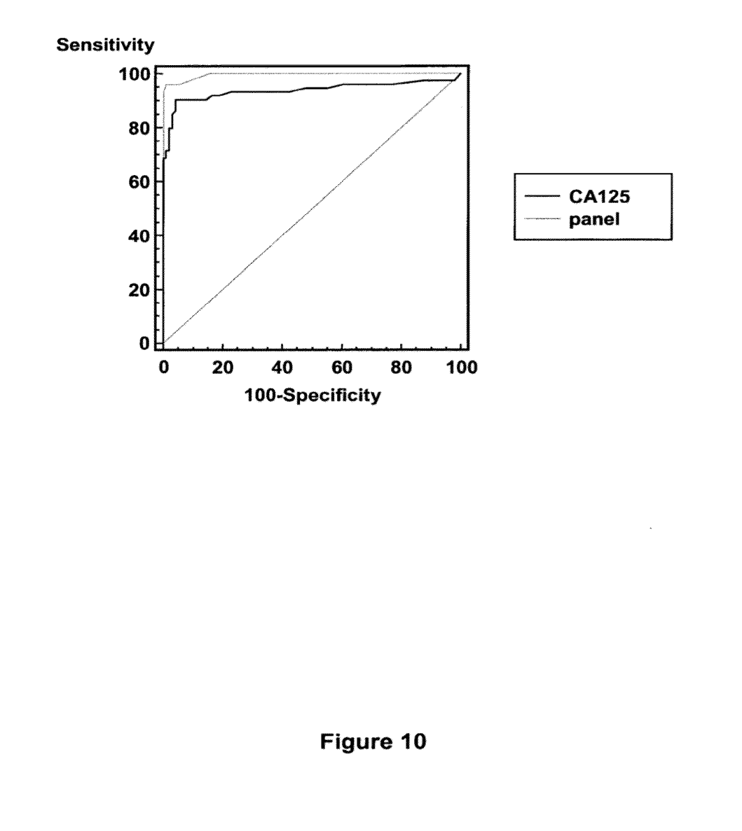

Further analysis was performed by testing for significant difference between the ROC curves for CA125 testing and those of the biomarker panel results (positive predictive value). The ROC curves were significantly different at the level of P=0.004, indicative of the superior performance of the biomarker panel over the CA 125 results alone (FIG. 10 and Table 10).

TABLE 10

| | AUC | SE | 95% CI |
|---|---|---|---|
| CA125 | 0.937 | 0.0206 | 0.890 to 0.969 |
| panel | 0.996 | 0.00546 | 0.970 to 0.999 |

Pairwise comparison of ROC curves
CA125~panel

| | |
|---|---|
| Difference between areas | 0.0582 |
| Standard error | 0.0204 |
| 95% Confidence interval | 0.0182 to 0.0982 |
| z statistic | 2.851 |
| Significance level | P = 0.004 |

To validate the performance of the biomarker panel the second sample subset, the validation set, were tested in the model algorithm. The ability to correctly classify each sample using the marker panel was assessed in terms of both sensitivity and specificity measures alongside CA125 alone, and also with regards to ROC analysis.

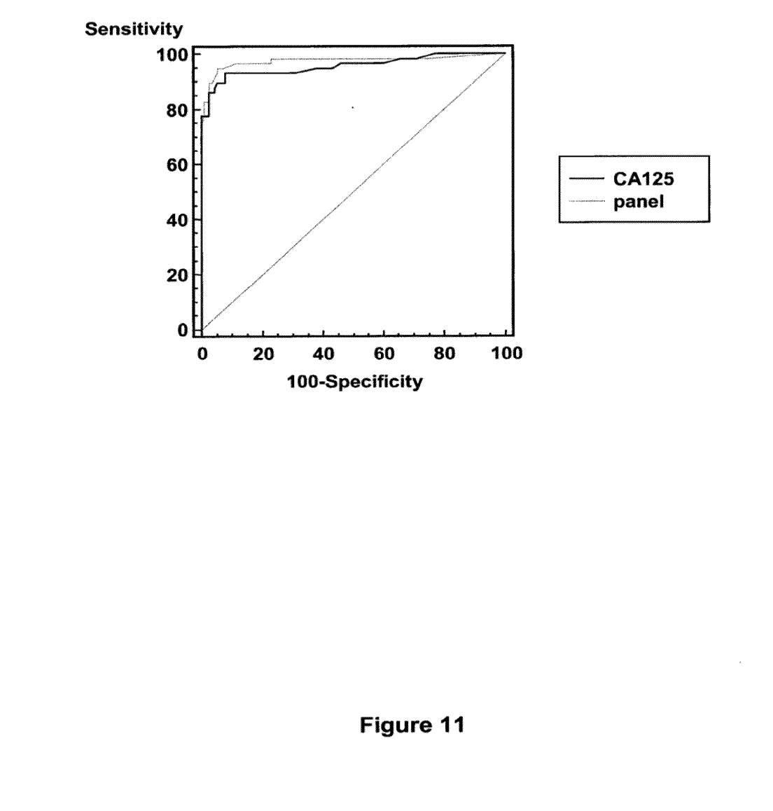

The validation sample subset as for modeling included only stage I, II and III disease levels and healthy controls. No stage IV or non-stage samples were included. In total 58 disease and 113 control samples were run through the model algorithm (Tables 11 and 12 and FIG. 11).

TABLE 11

| Diagnostic | False | False | True | True | | | Diagnostic |
|---|---|---|---|---|---|---|---|
| Test | negatives | positives | negatives | positives | Sensitivity | Specificity | Efficiency |
| CA125 | 4 | 12 | 101 | 54 | 93.1% | 89.4% | 91.25% |
| CA125/SAA/ | 3 | 6 | 107 | 55 | 94.8% | 94.7% | 94.75% |
| IL8/MK | | | | | | | |

TABLE 12

| | AUC | SE | 95% CI |
|---|---|---|---|
| CA125 | 0.956 | 0.0193 | 0.914 to 0.981 |
| panel | 0.975 | 0.0148 | 0.938 to 0.992 |

Pairwise comparison of ROC curves
CA125~panel

| | |
|---|---|
| Difference between areas | 0.0184 |
| Standard error | 0.0222 |
| 95% Confidence interval | −0.0251 to 0.0619 |
| z statistic | 0.829 |
| Significance level | P = 0.407 |

Finally, the total outcome for all samples was compared through the model by combining both model and validation results for comparison with CA 125.

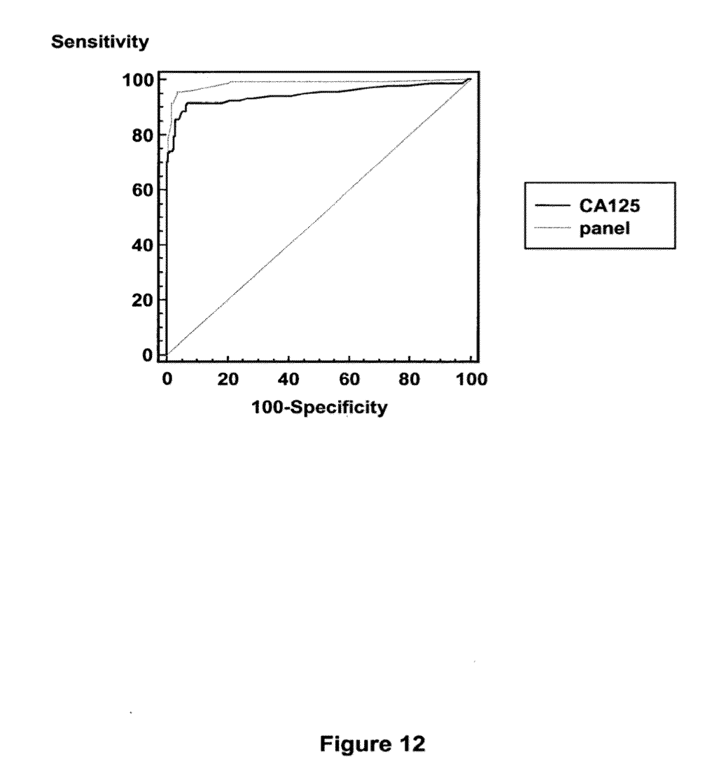

Thus, the total disease population is 132 and our total control population is 209 individuals (Tables 13 and 14 and FIG. 12).

TABLE 13

| Diagnostic | False | False | True | True | | | Diagnostic |
|---|---|---|---|---|---|---|---|
| Test | negatives | positives | negatives | positives | Sensitivity | Specificity | Efficiency |
| CA125 | 11 | 16 | 193 | 121 | 91.7% | 92.3% | 92.0% |
| CA125/SAA/ | 6 | 7 | 202 | 126 | 95.5% | 96.7% | 96.1% |
| IL8/MK | | | | | | | |

When the ROC curves were compared a significant improvement was found over CA125 alone in diagnosing ovarian cancer.

TABLE 14

| | AUC | SE | 95% CI |
|---|---|---|---|
| CA125 | 0.945 | 0.0143 | 0.916 to 0.967 |
| panel | 0.985 | 0.00746 | 0.966 to 0.995 |

Pairwise comparison of ROC curves
CA125~panel

| | |
|---|---|
| Difference between areas | 0.040 |
| Standard error | 0.015 |
| 95% Confidence interval | 0.0107 to 0.0694 |
| z statistic | 2.674 |
| Significance level | P = 0.008 |

Alternative algorithm modelings may be performed, e.g. bayesNET, NBTree, or AdaBoostM1. See Tables 15 and 16.

TABLE 15
For the modeling samples

| | | | Diagnostic | |
|---|---|---|---|---|
| Model | Sensitivity | Specificity | Efficiency | Area Under Curve |
| CA125 | 90.5% | 95.8% | 93.15% | 0.937 |
| bayesNET | 91.9% | 99.0% | 95.45% | 0.982 |
| NBTree | 93.2% | 99.0% | 96.1% | 0.961 |
| AdaBoostM1 | 91.9% | 99.0% | 95.45% | 0.991 |

TABLE 16
For the validations samples

| | | | Diagnostic | |
|---|---|---|---|---|
| Model | Sensitivity | Specificity | Efficiency | Area Under Curve |
| CA125 | 93.1% | 89.4% | 91.25% | 0.956 |
| bayesNET | 96.6% | 91.2% | 93.9% | 0.975 |
| NBTree | 93.1% | 95.6% | 94.35% | 0.963 |
| AdaBoostM1 | 93.1% | 95.6% | 94.35% | 0.970 |

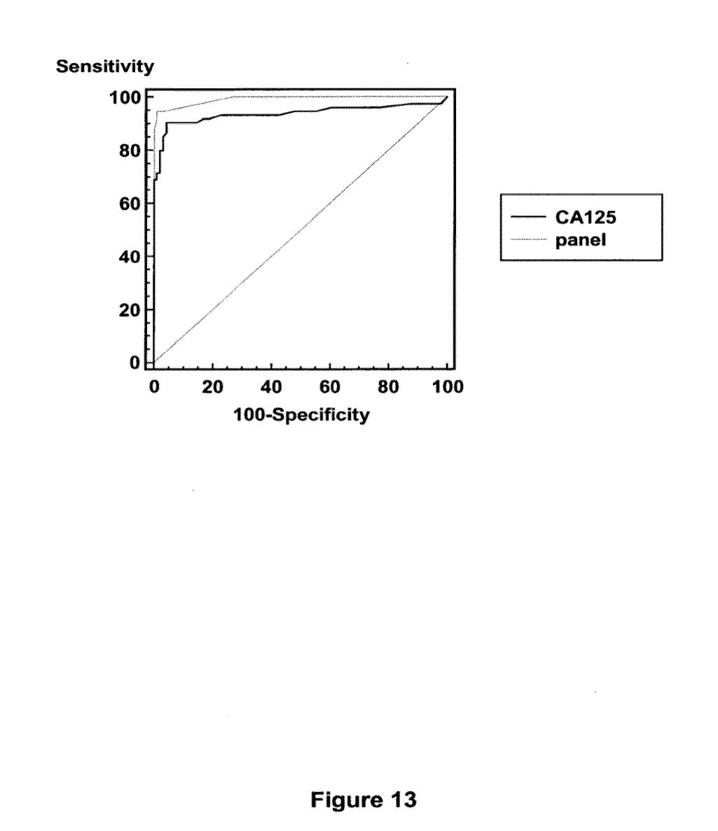

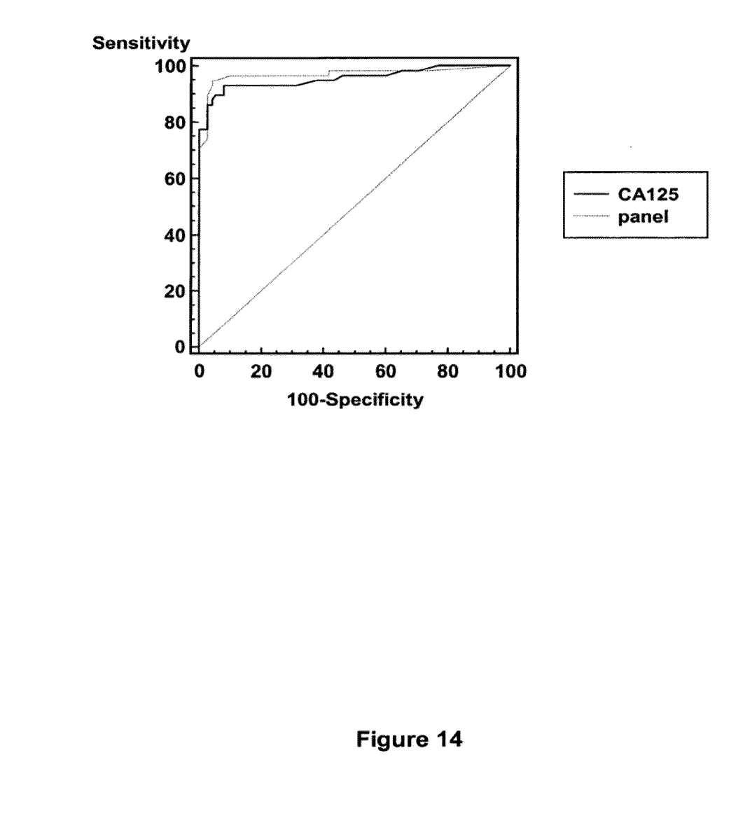

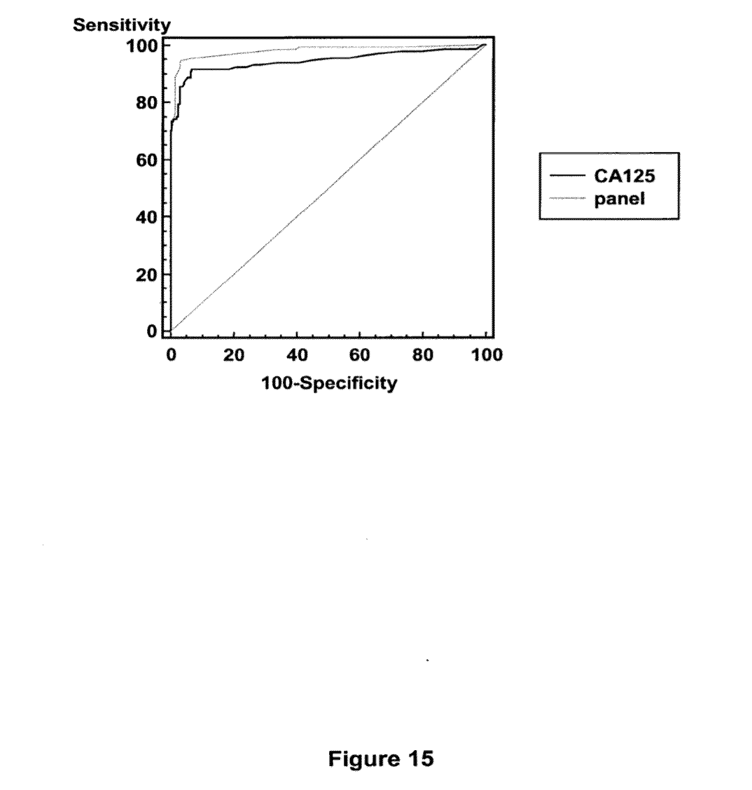

As an example of the above, the ROC curve comparison to CA125 is shown below for AdaBoostM1 algorithm modeling (Tables 17 to 19; FIGS. 13 to 15).

TABLE 17
Model set analysis.

| | AUC | SE | 95% CI |
|---|---|---|---|
| CA125 | 0.937 | 0.0206 | 0.890 to 0.969 |
| panel | 0.991 | 0.00769 | 0.963 to 0.999 |

Pairwise comparison of ROC curves
CA125~panel

| | |
|---|---|
| Difference between areas | 0.0539 |
| Standard error | 0.020 |
| 95% Confidence interval | 0.0146 to 0.0932 |
| z statistic | 2.690 |
| Significance level | P = 0.007 |

TABLE 18
Validation set analysis of ROC curves.

| | AUC | SE | 95% CI |
|---|---|---|---|
| CA125 | 0.956 | 0.0193 | 0.914 to 0.981 |
| panel | 0.970 | 0.016 | 0.932 to 0.990 |

Pairwise comparison of ROC curves
CA125~panel

| | |
|---|---|
| Difference between areas | 0.0138 |
| Standard error | 0.0213 |
| 95% Confidence interval | −0.028 to 0.0556 |
| z statistic | 0.647 |
| Significance level | P = 0.517 |

TABLE 19
Combined data set.

| | AUC | SE | 95% CI |
|---|---|---|---|
| CA125 | 0.945 | 0.0143 | 0.916 to 0.967 |
| panel | 0.980 | 0.00865 | 0.959 to 0.992 |

Pairwise comparison of ROC curves
CA125~panel

| | |
|---|---|
| Difference between areas | 0.0349 |
| Standard error | 0.0146 |
| 95% Confidence interval | 0.00634 to 0.0635 |
| z statistic | 2.394 |
| Significance level | P = 0.017 |

### Example 9

### AGR-2

Fourteen ovarian cancer samples, stage I and II only, were assayed alongside 16 female control plasma samples, in an ELISA developed for the detection of AGR-2.

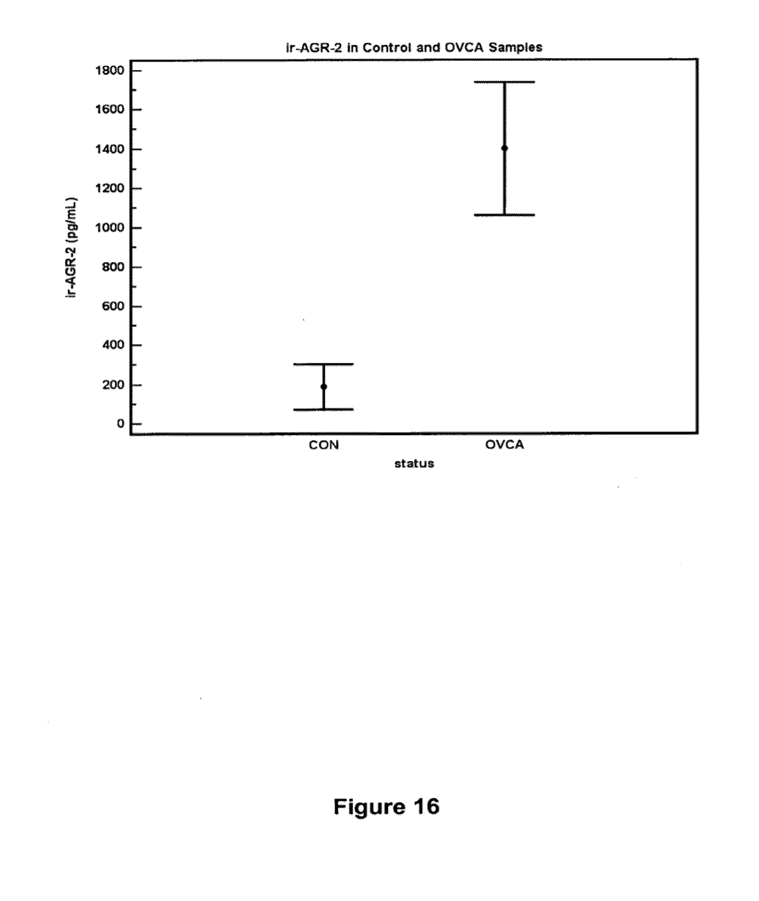

Results indicated that AGR-2 concentrations are elevated in plasma from early stage ovarian cancer patients as compared to control samples (FIG. 16).

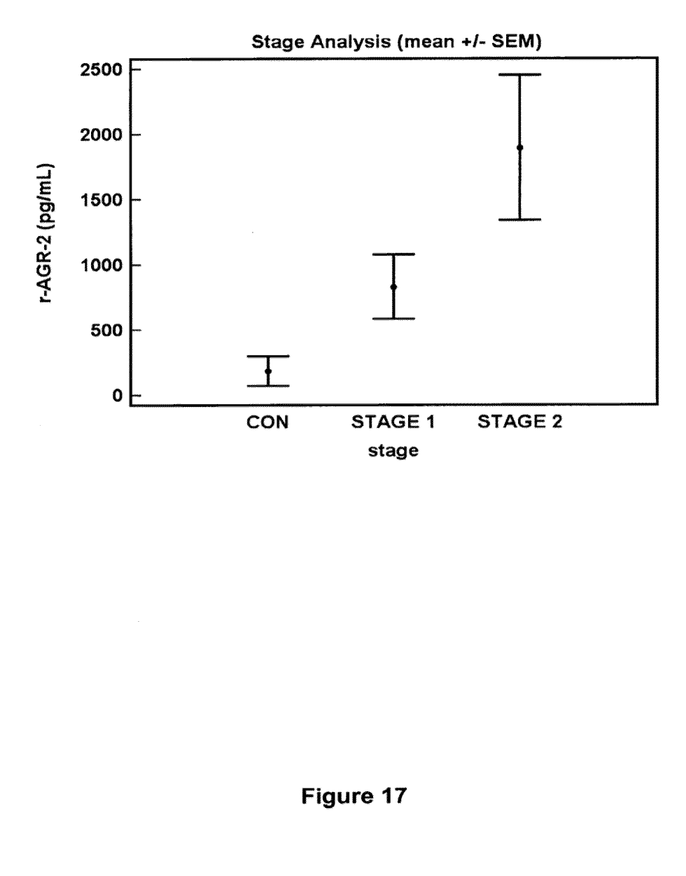

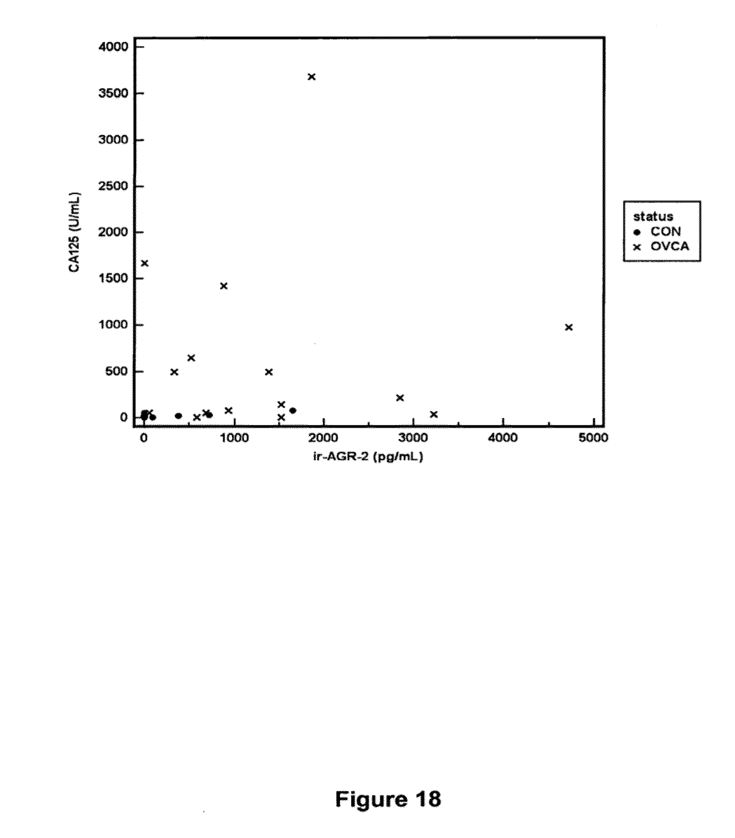

Furthermore, when the disease group is split according to stage, i.e. Stage I and Stage II disease there is indication that as the disease progresses the concentration of circulating plasma AGR-2 continues to rise (FIG. 17).

Correlation analysis indicated that there is not a direct correlation, i.e. linear relationship between AGR-2 and CA125, with a calculated correlation coefficient of 0.27.

The capacity to improve diagnosis using AGR-2 was determined by logitboost modeling using weka software. A model was built using two markers CA125 and AGR-2.

For analysis purposes CA125 analysis alone was based on a 35 unit clinical cut-off (Table 20).

TABLE 20

| Diagnostic | False | False | True | True | | | Diagnostic |
|---|---|---|---|---|---|---|---|
| Test | negatives | positives | negatives | positives | Sensitivity | Specificity | Efficiency |
| CA125 | 3 | 12 | 2 | 13 | 85.7% | 81.25% | 83.5% |
| CA125/AGR- | 1 | 14 | 0 | 15 | 100% | 93.75% | 96.7% |
| 2 panel | | | | | | | |
| CA125/AGR- | 0 | 14 | 0 | 16 | 100% | 100% | 100% |
| 2/MK panel | | | | | | | |

Further assessment of clinical potential was made by ROC plot analysis of CA 125 alongside AGR-2 alone, and also the posterior probability values determined by the modeled CA125/AGR-2 combination.

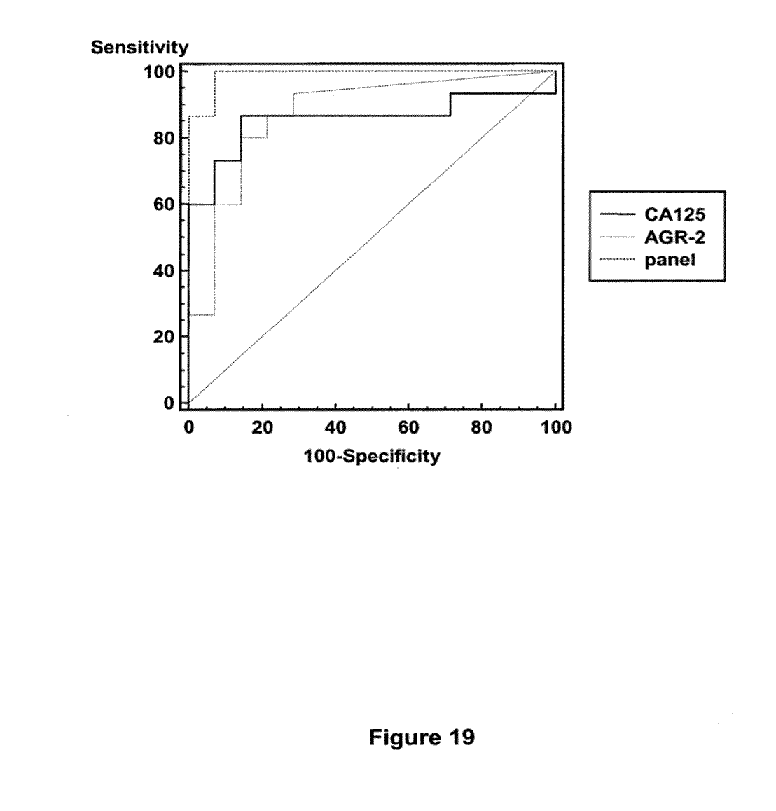

The ROC results indicate that the modeled CA125/AGR-2 provides superior clinical diagnostic performance to that of CA125 the recognized standard in ovarian cancer diagnostic testing (Table 21; FIG. 19).

TABLE 21

| | AUC | SE | 95% CI |
|---|---|---|---|
| CA125 | 0.857 | 0.0714 | 0.677 to 0.958 |
| AGR-2 | 0.871 | 0.0679 | 0.695 to 0.965 |
| panel | 0.990 | 0.0185 | 0.862 to 1.000 |

Pairwise comparison of ROC curves
CA125~AGR-2

| | |
|---|---|
| Difference between areas | 0.0143 |
| Standard error | 0.0931 |
| 95% Confidence interval | −0.168 to 0.197 |
| z statistic | 0.154 |
| Significance level | P = 0.878 |

CA125~panel

| | |
|---|---|
| Difference between areas | 0.133 |
| Standard error | 0.0691 |
| 95% Confidence interval | −0.00204 to 0.269 |
| z statistic | 1.931 |
| Significance level | P = 0.054 |

AGR-2~panel

| | |
|---|---|
| Difference between areas | 0.119 |
| Standard error | 0.0655 |
| 95% Confidence interval | −0.00942 to 0.248 |
| z statistic | 1.816 |
| Significance level | P = 0.069 |

Further modeling was performed to examine the utility of CA125, AGR-2 and midkine in combination. The result in this case was 100% sensitivity and specificity were achieved, with no false positives or false negatives, and an ROC value of 1.000 consequently.

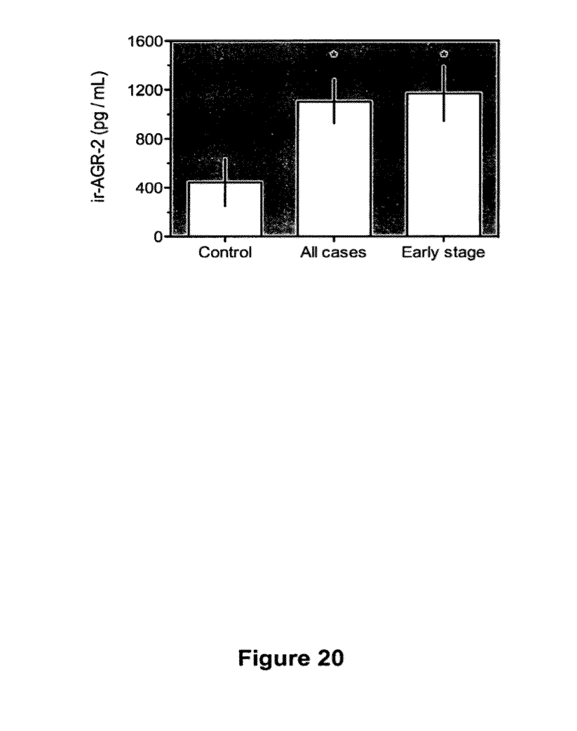

A second set of samples comprising 61 Control and 46 Ovarian Cancer (Stages I-III) patient plasma samples were assayed. The results confirm that plasma levels of AGR-2 are elevated in early stage ovarian cancer patients and remain elevated throughout the latter stages of disease. The changes in AGR2 in all ovarian cancer samples as well as early stage samples was shown to be significantly different to controls (Kruskal-Wallis non-parametric ANOVA followed by Dunn's Multiple Comparison Test (FIG. 20).

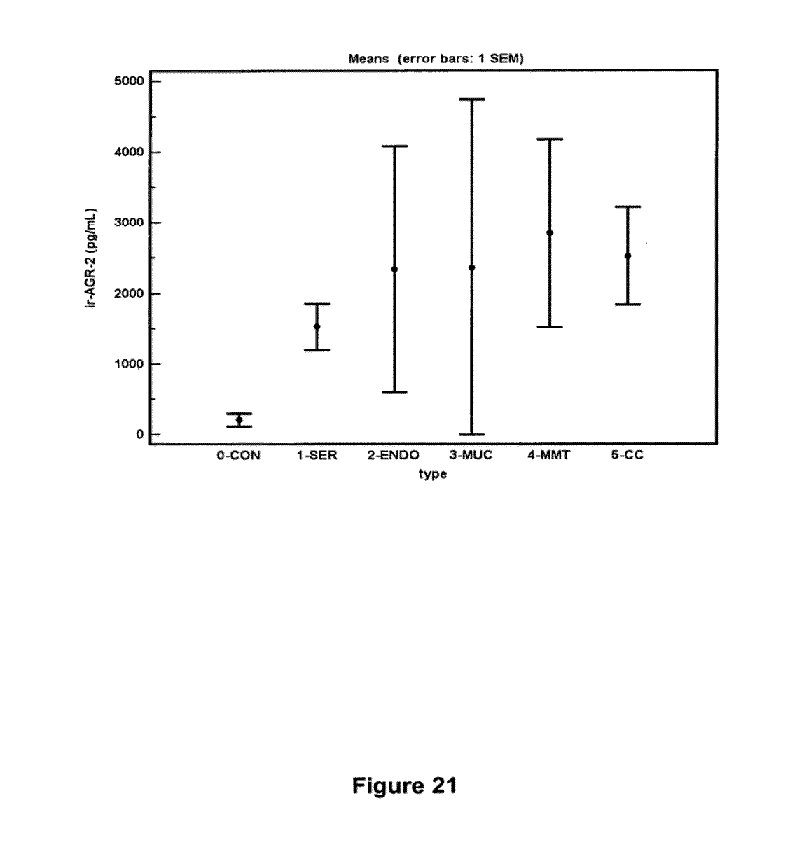

Plasma AGR-2 analysis according to disease type (FIG. 21) indicates that whereas CA125 is generally considered to be more useful in diagnosing serous type and lacks good diagnostic utility for other forms of OVCA disease, AGR-2 shows greatest elevation in the other forms of the disease.

### Example 10

### Midkine with CA125

Plasma samples were obtained from individuals with only stage I, II and III level disease. All patients with level IV disease were omitted as were those whose stage data was not available. Age matched controls were also assayed.

All patients and controls were randomly assigned to either modeling or validation data subsets, for the purpose of biomarker panel analysis.

Model set contained 74 disease and 96 controls. Of these 7 disease samples were negative by CA125 testing, having values lower than 35 U/ml. Of the controls 4 were given false positive results (e.g. values >/=35 U/ml) in CA125 testing.

Using logitboost modeling in weka software, a model was built. In this model only 1 control sample was given a false positive result, and 3 disease samples falsely assigned as negative for ovarian cancer (Table 22).

TABLE 22
With model set

| Diagnostic | False | False | True | True | | | Diagnostic |
|---|---|---|---|---|---|---|---|
| Test | negatives | positives | negatives | positives | Sensitivity | Specificity | Efficiency |
| CA125 | 7 | 4 | 92 | 67 | 90.5% | 95.8% | 93.15% |
| CA125/MK | 5 | 1 | 95 | 69 | 93.2% | 99.0% | 96.1% |

Further analysis was performed by testing for significant difference between the ROC curves for CA125 testing and those of the biomarker panel results (positive predictive value). The ROC curves were significantly different at the level of P=0.004, indicative of the superior performance of the biomarker panel over the CA125 results alone (FIG. 21).

### With Validation Set

To validate the performance of the biomarker panel the second sample subset, the validation set, were tested in the model algorithm. The ability to correctly classify each sample using the marker panel was assessed in terms of both sensitivity and specificity measures alongside CA125 alone, and also with regards to ROC analysis.

The validation sample subset as for modeling included only stage I, II and III disease levels and healthy controls. No stage IV or non-stage samples were included. In total 58 disease and 113 control samples were run through the model algorithm (Table 23).

TABLE 23

| Diagnostic | False | False | True | True | | | Diagnostic |
|---|---|---|---|---|---|---|---|
| Test | negatives | positives | negatives | positives | Sensitivity | Specificity | Efficiency |
| CA125 | 4 | 12 | 191 | 54 | 93.1% | 89.4% | 91.25% |
| CA125/MK | 7 | 6 | 107 | 51 | 87.9% | 94.7% | 91.3% |

### Combined Model + Validation Set

Finally, the total outcome was compared for all samples through the model by combining both model and validation results for comparison with CA125 (Table 24).

Thus, the total disease population is 132 and the total control population is 209 individuals.

TABLE 24

| Diagnostic | False | False | True | True | | | Diagnostic |
|---|---|---|---|---|---|---|---|
| Test | negatives | positives | negatives | positives | Sensitivity | Specificity | Efficiency |
| CA125 | 11 | 16 | 193 | 121 | 91.7% | 92.3% | 92.0% |
| CA125/MK | 12 | 7 | 202 | 120 | 90.9% | 96.7% | 93.8% |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Aberger et al, Mech Dev 72(1-2):115-130, 1998
Berek et al, Am J Obstet Gynecol. 164 (4):1038-42, 1991
Cooper et al, Clin Cancer Res. 8 (10):3193-7, 2002
Di Blasio et al, J Steroid Biochem Mol Biol. 53 (1-6):375-9, 1995
Gadducci et al, Anticancer Res 19 (2B):1401-5, 1999
Gorelik et al. Cancer Epidemiol, Biomarkers Prev 14(4):981-987, 2005
Holschneider and Berek, Semin Surg Oncol, 19 (1):3-10, 2000
Karayiannakis et al. Surgery 131 (5):548-55, 2002
Lee et al, Int J Oncol 17 (1):149-52, 2000
Liu et al, Cancer Res 65 (9):3796-3805, 2005
Oehler and Caffier, Anticancer Res. 20 (6D):5109-12, 2000
Sambrook et al, Molecular Cloning. A Laboratory Manual. (2nd ed.), 1989
Santin et al, Eur J Gynaecol Onco 20 (3):177-81, 1999
Senger et al, Science 219 (4587):983-5, 1983
Thompson and Weigel, Biochem Biophys Res Commun 251(1):111-116, 1998
Veikkola et al, Cancer Res 60 (2):203-12, 2000
Visintin et al, Clin Cancer Res 14(4):1065-1072, 2008

1. A method of allowing a user to determine the status of a subject with respect to a gynecological cancer or subtype thereof or stage of cancer, the method comprising:
(a) receiving data in the form of levels or concentrations of CA 125 and one or more of AGR-2, midkine, CRP, IL-6, IL-8, SAA and SAP from the user via a communications network;
(b) processing the subject data via a multivariate analysis to provide a disease index value;
(c) determining the status of the subject in accordance with the results of the disease index value in comparison with predetermined values; and
(d) transferring an indication of the status of the subject to the user via the communications network.

2-40. (canceled)
